(19) United States
(12) Patent Application Publication
(10) Pub. No.: US 2012/0165326 A1
(43) Pub. Date: Jun. 28, 2012

(54) DERIVATIVES OF 6-(6-SUBSTITUTED-TRIAZOLOPYRIDAZINE-SULFANYL) 5-FLUORO BENZOTHIAZOLES AND 5-FLUORO BENZIMIDAZOLES, PREPARATION THEREOF, USE THEREOF AS DRUGS, AND USE THEREOF AS MET INHIBITORS

(75) Inventors: Conception Nemecek, Paris (FR); Antonio Ugolini, Paris (FR); Eric Bacque, Paris (FR); Dominique Damour, Paris (FR); Gilles Barbalat, Paris (FR); Sylvie Wentzler, Paris (FR)

(73) Assignee: SANOFI, Paris (FR)

(21) Appl. No.: 13/147,644

(22) PCT Filed: Feb. 4, 2010
(86) PCT No.: PCT/FR10/50180

§ 371 Date: Mar. 15, 2012

(30) Foreign Application Priority Data
Feb. 6, 2009 (FR) 0900514

(51) Int. Cl. A61K 31/5025; A61K 31/5377; C07D 277/82; A61P 35/00; A61P 9/00; A61P 3/00; A61P 37/08; A61P 11/06; A61P 7/02; A61P 25/00; A61P 9/10; A61P 3/10; A61P 21/00; A61P 17/06; A61P 19/02; A61P 35/04; C07D 417/12; A61P 29/00; C07D 487/04

(52) U.S. Cl. 514/233.2; 544/118; 544/236; 514/248; 548/164; 548/163; 544/368

(57) ABSTRACT

The invention relates to novel products of the formula (I) where: (II) is a single or double bond; F is a fluorine atom, Ra is H, HaI, alkoxy, O-cycloalkyl, —O— heterocycloalkyl; —NH-heterocycloalkyl, heteroaryl, phenyl, NHCOalk NHCOcycloalk or NR1 R2; X is S, SO or SO2, A is NH or S; W is H, alkyl, or COR with R being cycloalkyl; alkyl optionally substituted by NR3R4, alkoxy, hydroxy, phenyl, heteroaryl or heterocycloalkyl; alkoxy optionally substituted by NR3R4, i.e. a O—(CH2)n-NR3R4 radical, an O-phenyl or an O—(CH2)n-phenyl radical, with phenyl optionally substituted and n=1 to 4; or the NR1 R2 radical; R1 is H or alk and R2 is H, cycloalkyl or alkyl; R3 and R4 are H, alk, cycloalkyl, heteroaryl or phenyl; R1, R2 and/or R3, R4 form a cycle together with N optionally containing O, S, N and/or NH; heterocycloalkyl, heteroaryl and phenyl and cycles all being optionally substituted; wherein said products can be in any isomer or salt form, and can be used as drugs, in particular as MET inhibitors.

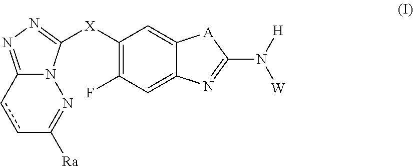

The present invention relates to novel 6-(6-substituted triazolopyridazine-sulfanyl) 5-fluorobenzothiazole and 5-fluorobenzimidazole derivatives, to a process for preparing them, to the novel intermediates obtained, to their use as medicaments, to pharmaceutical compositions containing them and to the novel use of such 6-(6-substituted triazolopyndazine-sulfanyl) 5-fluorobenzothiazole and 5-fluorobenzimidazole derivatives.

The present invention more particularly relates to novel 6-(6-substituted triazolopyridazine-sulfanyl) 5-fluorobenzothiazole and 5-fluorobenzimidazole derivatives with anticancer activity, via modulation of the activity of proteins, in particular kinases.

To date, most of the commercial compounds used in chemotherapy are cytotoxic and pose major problems of side effects and tolerance by patients. These effects can be limited if the medicaments used act selectively on cancer cells, to the exclusion of healthy cells. One of the solutions for limiting the undesirable effects of a chemotherapy may thus consist in using medicaments that act on metabolic pathways or constituent elements of these pathways, predominantly expressed in cancer cells, and not expressed or only sparingly expressed in healthy cells. Kinase proteins are a family of enzymes that catalyse the phosphorylation of hydroxyl groups of specific protein residues such as tyrosine, serine or threonine residues. Such phosphorylations may widely modify the function of proteins: thus, kinase proteins play an important role in regulating a wide variety of cellular processes, especially including cell metabolism and proliferation, cellular adhesion and motility, cell differentiation or cell survival, certain kinase proteins playing a central role in the initiation, development and completion of cell cycle events.

Among the various cellular functions in which the activity of a kinase protein is involved, certain processes represent attractive targets for treating certain diseases. Examples that may especially be mentioned include angiogenesis and control of the cell cycle and also that of cell proliferation, in which kinase proteins may play an essential role. These processes are especially essential for the growth of solid tumours and also other diseases: in particular, molecules that inhibit such kinases are capable of limiting undesired cell proliferations such as those observed in cancers, and can intervene in the prevention, regulation or treatment of neurodegenerative diseases such as Alzheimer's disease or neuronal apoptosis.

One subject of the present invention is novel derivatives endowed with inhibitory effects on kinase proteins. The products according to the present invention may thus be used especially for preventing or treating diseases that can be modulated by inhibiting kinase proteins.

The products according to the present invention especially show anticancer activity, via modulation of the activity of kinases. Among the kinases for which activity modulation is desired, MET and also mutants of the protein MET are preferred.

The present invention also relates to the use of the said derivatives for preparing a medicament for treating man.

Thus, one of the objects of the present invention is to propose compositions with anticancer activity, by acting in particular on kinases. Among the kinases for which activity modulation is desired, MET is preferred.

In the pharmacological section hereinbelow, it is shown in biochemical tests and on cell lines that the products of the present patent application thus especially inhibit the autophosphorylation activity of MET and the proliferation of cells whose growth is dependent on MET or mutants forms thereof.

MET, or Hepatocyte Growth Factor Receptor, is a receptor with tyrosine kinase activity that is expressed in particular in epithelial and endothelial cells. HGF, Hepatocyte Growth Factor, is described as the specific ligand of MET. HGF is secreted by mesenchymal cells and activates the MET receptor, which homodimerizes. Consequently, the receptor becomes autophosphorylated on tyrosines Y1230, Y1234 and Y1235 of the catalytic domain.

Stimulation of MET with HGF induces cell proliferation, scattering (or dispersion) and motility, resistance to apoptosis, invasion and angiogenesis.

MET and likewise HGF are found to be overexpressed in many human tumours and a wide variety of cancers. MET is also found to be amplified in gastric tumours and glioblastomas. Many point mutations of the MET gene have also been described in tumours, in particular in the kinase domain, but also in the juxtamembrane domain and the SEMA domain. Overexpression, amplification or mutations cause constitutive activation of the receptor and deregulation of its functions.

The present invention thus relates especially to novel inhibitors of the kinase protein MET and mutants thereof, which may be used for anti-proliferative and anti-metastatic treatment especially in oncology.

The present invention also relates to novel inhibitors of the kinase protein MET and mutants thereof, which may be used for anti-angiogenic treatment, especially in oncology.

One subject of the present invention is the products of formula (I):

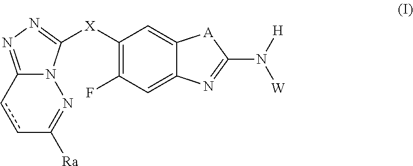

in which [symbol]
represents a single or double bond;
Ra represents a hydrogen atom; a halogen atom; an alkoxy radical optionally substituted with a chlorine atom, a hydroxyl radical or a heterocycloalkyl radical, which is itself optionally substituted; a radical —O-cycloalkyl, —O-heterocycloalkyl; —NH-cycloalkyl and —NH-heterocycloalkyl, all optionally substituted; an optionally substituted heteroaryl radical; an optionally substituted phenyl radical; a radical NHCOalkyl or NHCOcycloalkyl; or a radical NR1R2 as defined below;
X represents S, SO or SO2;
A represents NH or S;
W represents a hydrogen atom; an alkyl, cycloalkyl or heterocycloalkyl radical optionally substituted with alkoxy, heterocycloalkyl or NR3R4; or the radical COR in which R represents:
a cycloalkyl radical or an alkyl radical, optionally substituted with a radical NR3R4, alkoxy, hydroxyl, phenyl, heteroaryl or heterocycloalkyl, which are themselves optionally substituted;
an alkoxy radical optionally substituted with NR3R4, alkoxy, hydroxyl or heterocycloalkyl; a radical O-phenyl or a radical O—(CH2)ₙ-phenyl, with phenyl optionally substituted and n represents an integer from 1 to 4;
or the radical NR1R2 in which R1 and R2 are such that one from among R1 and R2 represents a hydrogen atom or an alkyl radical and the other from among R1 and R2 represents a hydrogen atom, a cycloalkyl radical or an alkyl radical optionally substituted with one or more radicals, which may be identical or different, chosen from the following radicals: hydroxyl, alkoxy, heteroaryl, heterocycloalkyl, NR3R4, phenyl, optionally substituted or alternatively R1 and R2 form, with the nitrogen atom to which they are attached, a 3- to 10-membered cyclic radical optionally containing one or more other heteroatoms chosen from O, S, N and NH, including the possible NH it contains, being optionally substituted;
with R3 and R4, which may be identical or different, representing a hydrogen atom, an alkyl radical, a cycloalkyl radical, a heteroaryl radical or a phenyl radical, all optionally substituted with one or more radicals, which may be identical or different, chosen from the following radicals: hydroxyl, alkoxy, heteroaryl, heterocycloalkyl, NH2, NHAlk, N(Alk)2 or phenyl, optionally substituted; or alternatively R3 and R4 form, with the nitrogen atom to which they are attached, a 3- to 10-membered cyclic radical optionally containing one or more other heteroatoms chosen from O, S, N and NH, this radical, including the possible NH it contains, being optionally substituted;
   all the alkyl, cycloalkyl, heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms and the following radicals: hydroxyl, oxo, alkoxy, NH2, NHalk, N(alk)2 and alkyl, cycloalkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and the following radicals: hydroxyl, oxo, alkyl and alkoxy containing from 1 to 4 carbon atoms, NH2, NHalk and N(alk)2;
   the said products of formula (I) being in any possible racemic, enantiomeric or diastereoisomeric isomer form, and also the addition salts with mineral and organic acids or with mineral and organic bases of the said products of formula (I).

In the products of formula (I), F represents a fluorine atom.

The present invention relates especially to the products of formula (I) in which Ra represents a hydrogen atom; a halogen atom; an alkoxy radical optionally substituted with a heterocycloalkyl radical, which is itself optionally substituted; an optionally substituted heteroaryl radical; an optionally substituted phenyl radical; an optionally substituted radical —O-cycloalkyl; optionally substituted O-heterocycloalkyl; optionally substituted —NH-cycloalkyl; optionally substituted —NH-heterocycloalkyl; a radical —NHCOalkyl or —NHCOcycloalkyl; or a radical NR1R2 as defined hereinabove or hereinbelow, the other substituents on the said products of formula (I) having any of the meanings indicated hereinabove or hereinbelow.

A subject of the present invention is the products of formula (I) as defined hereinabove or hereinbelow, in which [symbol] , X and A have the meanings given hereinabove or hereinbelow,

Ra represents an alkoxy radical optionally substituted with a chlorine atom, a hydroxyl radical or a heterocycloalkyl radical, which is itself optionally substituted; a radical —O-cycloalkyl, a radical —NHCOalk; or a radical —NR1aR2a; such that R1a and R2a represent a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and phenyl radicals, optionally substituted;
and W represents a hydrogen atom; an alkyl radical optionally substituted with alkoxy, heterocycloalkyl or NR3R4; or the radical COR in which R represents:
a cycloalkyl radical or an alkyl radical, optionally substituted with a radical NR3R4, alkoxy, hydroxyl, phenyl, heteroaryl or heterocycloalkyl, which are themselves optionally substituted;
an alkoxy radical optionally substituted with NR3R4, alkoxy, hydroxyl or heterocycloalkyl; a radical O-phenyl or a radical O—(CH2)n-phenyl, with phenyl optionally substituted and n represents an integer from 1 to 4;
or the radical NR1R2 in which R1 and R2 are such that one from among R1 and R2 represents a hydrogen atom or an alkyl radical and the other from among R1 and R2 represents a hydrogen atom, a cycloalkyl radical or an alkyl radical optionally substituted with one or more radicals, which may be identical or different, chosen from the following radicals: hydroxyl, alkoxy, heteroaryl, heterocycloalkyl, NR3R4, phenyl, optionally substituted or alternatively R1 and R2 form, with the nitrogen atom to which they are attached, a 3- to 10-membered cyclic radical optionally containing one or more other heteroatoms chosen from O, S, N and NH, this radical, including the possible NH it contains, being optionally substituted;
   with R3 and R4, which may be identical or different, representing a hydrogen atom, an alkyl radical, a cycloalkyl radical, a heteroaryl radical or a phenyl radical all optionally substituted with one or more radicals, which may be identical or different, chosen from the following radicals: hydroxyl, alkoxy, heteroaryl, heterocycloalkyl, NH2, NHAlk, N(Alk)2 or phenyl, optionally substituted; or alternatively R3 and R4 form, with the nitrogen atom to which they are attached, a 3- to 10-membered cyclic radical optionally containing one or more other heteroatoms chosen from O, S, N and NH, this radical, including the possible NH it contains, being optionally substituted;
   all the heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms and the following radicals: hydroxyl, oxo, alkoxy, NH2, NHalk, N(alk)2 and alkyl, cycloalkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and the following radicals: hydroxyl, oxo, alkyl and alkoxy containing from 1 to 4 carbon atoms, NH2, NHalk and N(alk)2;
   the said products of formula (I) being in any possible racemic, enantiomeric or diastereoisomeric isomer form, and also the addition salts with mineral and organic acids or with mineral and organic bases of the said products of formula (I).

A subject of the present invention is the products of formula (I) as defined hereinabove or hereinbelow, in which [symbol] , X and A have the meanings given hereinabove or hereinbelow;

Ra represents an alkoxy radical optionally substituted with a heterocycloalkyl radical, which is itself optionally substituted; a radical NHCOalk or a radical NR1aR2a; such that R1a and R2a represent a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and phenyl radicals, optionally substituted;
and W represents a hydrogen atom; an alkyl radical optionally substituted with alkoxy, heterocycloalkyl or NR3R4; or the radical COR in which R represents:

a cycloalkyl radical or an alkyl radical optionally substituted with a radical NR3R4, alkoxy, hydroxyl, phenyl, heteroaryl or heterocycloalkyl, which are themselves optionally substituted;

an alkoxy radical optionally substituted with NR3R4, alkoxy, hydroxyl or heterocycloalkyl; a radical O-phenyl or a radical O—(CH2)n-phenyl, with phenyl optionally substituted and n represents an integer from 1 to 4;

or the radical NR1R2, in which R1 and R2 are such that one from among R1 and R2 represents a hydrogen atom or an alkyl radical and the other from among R1 and R2 represents a hydrogen atom or an alkyl radical optionally substituted with one or more radicals, which may be identical or different, chosen from hydroxyl, alkoxy, heteroaryl, heterocycloalkyl, NR3R4 and phenyl radicals, optionally substituted, or alternatively R1 and R2 form, with the nitrogen atom to which they are attached, a 3- to 10-membered cyclic radical optionally containing one or more other heteroatoms chosen from O, S, N and NH, this radical, including the possible NH it contains, being optionally substituted;

with R3 and R4, which may be identical or different, represent a hydrogen atom, an alkyl radical, a cycloalkyl radical, a heteroaryl radical or a phenyl radical, all optionally substituted with one or more radicals, which may be identical or different, chosen from hydroxyl, alkoxy, heteroaryl or heterocycloalkyl radicals or NH2, NHAlk, N(Alk)2, or alternatively R3 and R4 form, with the nitrogen atom to which they are attached, a 3- to 10-membered cyclic radical optionally containing one or more other heteroatoms chosen from O, S, N and NH, this radical, including the possible NH it contains, being optionally substituted;
all the heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms and the following radicals: hydroxyl, oxo, alkoxy, NH2, NHalk, N(alk)2 and alkyl, cycloalkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and hydroxyl, oxo, alkyl and alkoxy radicals containing from 1 to 4 carbon atoms, NH2, NHalk and N(alk)2;
the said products of formula (I) being in any possible racemic, enantiomeric or diastereoisomeric isomer form, and also the addition salts with mineral and organic acids or with mineral and organic bases of the said products of formula (I).

One subject of the present invention is the products of formula (I) as defined above in which

[symbol]

represents a single or double bond;
Ra represents a hydrogen atom; a halogen atom, an alkoxy radical optionally substituted with a heterocycloalkyl radical, which is itself optionally substituted; an optionally substituted heteroaryl radical; an optionally substituted phenyl radical; a radical NHCOalkyl or NHCOcycloalkyl; or a radical NR1R2 as defined hereinbelow;
X represents S, SO or SO2;
A represents NH or S;
W represents a hydrogen atom; an alkyl radical optionally substituted with alkoxy, heterocycloalkyl or NR3R4; or the radical COR in which R represents:
a cycloalkyl radical or an alkyl radical, optionally substituted with a radical NR3R4, alkoxy, hydroxyl, phenyl, heteroaryl or heterocycloalkyl, which are themselves optionally substituted;
an alkoxy radical optionally substituted with NR3R4, alkoxy, hydroxyl or heterocycloalkyl; a radical O-phenyl or a radical O—(CH2)n-phenyl, with phenyl optionally substituted and n represents an integer from 1 to 4;
or the radical NR1R2 in which R1 and R2 are such that one from among R1 and R2 represents a hydrogen atom or an alkyl radical and the other from among R1 and R2 represents a hydrogen atom, a cycloalkyl radical or an alkyl radical optionally substituted with one or more radicals, which may be identical or different, chosen from the following radicals: hydroxyl, alkoxy, heteroaryl, heterocycloalkyl, NR3R4, phenyl, optionally substituted or alternatively R1 and R2 form, with the nitrogen atom to which they are attached, a 3- to 10-membered cyclic radical optionally containing one or more other heteroatoms chosen from O, S, N and NH, this radical, including the possible NH it contains, being optionally substituted;
   with R3 and R4, which may be identical or different, representing a hydrogen atom, an alkyl radical, a cycloalkyl radical, a heterocycloalkyl radical, a heteroaryl radical or a phenyl radical, all optionally substituted with one or more radicals, which may be identical or different, chosen from hydroxyl, alkoxy, heteroaryl, heterocycloalkyl, NH2, NHalk, N(alk)2 and phenyl radicals, optionally substituted, or alternatively R3 and R4 form, with the nitrogen atom to which they are attached, a 3- to 10-membered cyclic radical optionally containing one or more other heteroatoms chosen from O, S, N and NH, this radical, including the possible NH it contains, being optionally substituted;
   all the heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms and the following radicals: hydroxyl, oxo, alkoxy, NH2, NHalk, N(alk)2 and alkyl, cycloalkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and the following radicals: hydroxyl, oxo, alkyl and alkoxy containing from 1 to 4 carbon atoms, NH2, NHalk and N(alk)2,
   the said products of formula (I) being in any possible racemic, enantiomeric or diastereoisomeric isomer form, and also the addition salts with mineral and organic acids or with mineral and organic bases of the said products of formula (I).

The present invention, which thus concerns the products of formula (I) as defined above, in which [symbol] represents a single or double bond, thus precisely concerns the products of formula (I′) which represent the products of formula (I) in which [symbol] represents a single bond and the products of formula (I″) which represent the products of formula (I) in which [symbol] represents a double bond.

Thus, all the products of formula (I) as defined hereinabove or hereinbelow particularly represent products of formula (I′) in which [symbol] represents a single bond.

The products of formula (I) as defined hereinabove or hereinbelow also represent products of formula (I″) in which [symbol] represents a double bond.

A subject of the present invention is the products of formula (I) as defined hereinabove or hereinbelow, in which [symbol] , Ra and X have the values defined hereinabove or hereinbelow, and:

A represents NH or S;
W represents a hydrogen atom; an alkyl radical optionally substituted with alkoxy or heterocycloalkyl; or the radical COR in which R represents:

a cycloalkyl radical or an alkyl radical optionally substituted with a radical NR3R4, alkoxy, hydroxyl, phenyl, heteroaryl or heterocycloalkyl, which are themselves optionally substituted;

an alkoxy radical optionally substituted with NR3R4, alkoxy, hydroxyl or heterocycloalkyl; a radical O-phenyl or a radical O—(CH2)n-phenyl, with phenyl optionally substituted and n represents an integer from 1 to 4;

or the radical NR1R2, in which R1 and R2 are such that one from among R1 and R2 represents a hydrogen atom or an alkyl radical and the other from among R1 and R2 represents a hydrogen atom, a cycloalkyl radical or an alkyl radical optionally substituted with NR3R4 or alkoxy; or alternatively R1 and R2 form, with the nitrogen atom to which they are attached, a 3- to 10-membered cyclic radical optionally containing one or more other heteroatoms chosen from O, S, N and NH, this radical, including the possible NH it contains, being optionally substituted;

with NR3R4 such that R3 and R4, which may be identical or different, represent a hydrogen atom or an alkyl radical or alternatively R3 and R4 form, with the nitrogen atom to which they are attached, a 3- to 10-membered cyclic radical optionally containing one or more other heteroatoms chosen from O, S, N and NH, this radical, including the possible NH it contains, being optionally substituted;
all the heterocycloalkyl, heteroaryl and phenyl radicals, and also the cyclic radicals that may be formed by R1 and R2 or R3 and R4 with the nitrogen atom to which they are attached, defined above, being optionally substituted with one or more radicals chosen from halogen atoms and the following radicals: hydroxyl, alkoxy, NH2, NHalk, N(alk)2 and alkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and hydroxyl, alkyl and alkoxy radicals containing from 1 to 4 carbon atoms, NH2, NHalk and N(alk)2;
the said products of formula (I) being in any possible racemic, enantiomeric or diastereolsomeric isomer form, and also the addition salts with mineral and organic acids or with mineral and organic bases of the said products of formula (I),

One subject of the present invention is thus the products of formula (I) as defined above, in which [symbol] , Ra, X, A and W have any of the values defined hereinabove or hereinbelow, and the radical NR1R2 is such that one from among R1 and R2 represents a hydrogen atom or an alkyl radical, and the other from among R1 and R2 represents a hydrogen atom or an alkyl radical optionally substituted with NR3R4 or with alkoxy, or alternatively R1 and R2 form, with the nitrogen atom to which they are attached, a 3- to 10-membered cyclic radical optionally containing one or more other heteroatoms chosen from O, S, N and NH, this radical, including the optional NH it contains, being optionally substituted;

all the other substituents having the definitions given hereinabove;
the said products of formula (I) being in any possible racemic, enantiomeric or diastereoisomeric isomer form, and also the addition salts with mineral and organic acids or with mineral and organic bases of the said products of formula (I).

A subject of the present invention is the products of formula (I) as defined hereinabove or hereinbelow

in which [symbol]
represents a single or double bond
Ra represents a hydrogen atom or a halogen atom, or alternatively an optionally substituted phenyl radical,
X represents S, SO or SO₂
A represents NH or S;
W represents a hydrogen atom or a radical COR in which R represents:
a cycloalkyl radical or an alkyl radical optionally substituted with a phenyl, heteroaryl, NR3R4 or heterocycloalkyl radical, which are themselves optionally substituted;
an alkoxy radical optionally substituted with NR3R4, i.e. a radical O—(CH2)n—NR3R4; a radical O-phenyl or a radical O—(CH2)n-phenyl, with phenyl optionally substituted and n representing an integer from 1 to 4;
or the radical NR1R2 in which R1 and R2 are such that one from among R1 and R2 represents a hydrogen atom or an alkyl radical and the other from among R1 and R2 represents a cycloalkyl radical or an alkyl radical optionally substituted with one or more radicals, which may be identical or different, chosen from hydroxyl, alkoxy, heteroaryl, heterocycloalkyl, NR3R4 or phenyl radicals, optionally substituted, or alternatively R1 and R2 form, with the nitrogen atom to which they are attached, a cyclic radical optionally containing one or more other heteroatoms chosen from O, S, N and NH, this radical, including the optional NH it contains, being optionally substituted;
   with R3 and R4, which may be identical or different, representing a hydrogen atom, an alkyl radical, a cycloalkyl radical, a heteroaryl radical or a phenyl radical, optionally substituted, or alternatively R3 and R4 form, with the nitrogen atom to which they are attached, a cyclic radical optionally containing one or more other heteroatoms chosen from O, S, N and NH, this radical, including the optional NH it contains, being optionally substituted;
   all the heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms, hydroxyl, oxo, alkoxy, NH2, NHalk and N(alk)2 radicals and alkyl, cycloalkyl, CH2-heterocycloalkyl, CH2-phenyl, CO-phenyl and S-heteroaryl radicals, such that, in the latter radicals, the alkyl, heterocycloalkyl, phenyl and heteroaryl radicals are themselves optionally substituted with one or more radicals chosen from halogen atoms and hydroxyl, oxo, alkyl and alkoxy radicals containing from 1 to 4 carbon atoms, NH2, NHalk and N(alk)2,
   the said products of formula (I) being in any possible racemic, enantiomeric or diastereoisomeric isomer form, and also the addition salts with mineral and organic acids or with mineral and organic bases of the said products of formula (I).

Products of formula (I) as defined hereinabove or hereinbelow, in which [symbol] , Ra and X have the values defined hereinabove or hereinbelow, and:

A represents NH or S;
W represents a hydrogen atom or an alkyl radical or the radical COR in which R represents;

an alkyl radical optionally substituted with OCH3 or NR3R4;

a cycloalkyl radical

an alkoxy radical optionally substituted with OCH3 or NR3R4, i.e. a radical O—(CH2)n-OCH3 or a radical O—(CH2)n—NR3R4, a radcial O-phenyl or O—(CH2)n-phenyl, with phenyl optionally substituted and n represents an integer from 1 to 2;

or the radical NR1R2, in which R1 and R2 are such that one from among R1 and R2 represents a hydrogen atom, a cycloalkyl radical or an alkyl radical and the other from among R1 and R2 represents an alkyl radical optionally substituted with NR3R4, or alternatively R1 and R2 form, with the nitrogen atom to which they are attached, a cyclic radical optionally containing one or more other heteroatoms chosen from O, S, N and NH, this radical, including the possible NH it contains, being optionally substituted;

with NR3R4 such that R3 and R4, which may be identical or different, represent a hydrogen atom or an alkyl radical or alternatively R3 and R4 form, with the nitrogen atom to which they are attached, a cyclic radical optionally containing one or more other heteroatoms chosen from O, S, N and NH, this radical, including the possible NH it contains, being optionally substituted;
the phenyl radicals, and also the cyclic radicals that may be formed by R1 and R2 or R3 and R4 with the nitrogen atom to which they are attached, defined above, being optionally substituted with one or more radicals chosen from halogen atoms and the following radicals: hydroxyl, alkoxy, NH2, NHalk, N(alk)2 and alkyl, CH2-heterocycloalkyl, CH2-phenyl, CO-phenyl and S-heteroaryl radicals, such that, in the latter radicals, the alkyl, heterocycloalkyl, phenyl and heteroaryl radicals are themselves optionally substituted with one or more radicals chosen from halogen atoms and hydroxyl, alkyl and alkoxy radicals containing from 1 to 4 carbon atoms, NH2, NHalk and N(alk)2;
the said products of formula (I) being in any possible racemic, enantiomeric or diastereoisomeric isomer form, and also the addition salts with mineral and organic acids or with mineral and organic bases of the said products of formula (I).

One subject of the present invention is thus the products of formula (I) as defined above, in which [symbol] , Ra and X have any one of the values defined hereinabove or hereinbelow,

A represents NH or S;
W represents a hydrogen atom or the radical COR in which R represents:

an alkyl radical optionally substituted with NR3R4;

an alkoxy radical optionally substituted with NR3R4, i.e. a radical O—(CH2)n—NR3R4, a radical O-phenyl or O—(CH2)n-phenyl, with phenyl optionally substituted and n representing an integer from 1 to 2;

or the radical NR1R2, in which R1 and R2 are such that one from among R1 and R2 represents a hydrogen atom or an alkyl radical and the other from among R1 and R2 represents an alkyl radical optionally substituted with NR3R4, or alternatively R1 and R2 form, with the nitrogen atom to which they are attached, a cyclic radical optionally containing one or more other heteroatoms chosen from O, S, N and NH, this radical, including the optional NH it contains, being optionally substituted;

with NR3R4 such that R3 and R4, which may be identical or different, represent a hydrogen atom or an alkyl radical, or alternatively R3 and R4 form, with the nitrogen atom to which they are attached, a cyclic radical optionally containing one or more other heteroatoms chosen from O, S, N and NH, this radical, including the optional NH it contains, being optionally substituted;
the phenyl radicals and the cyclic radicals that may be formed by R1 and R2 or R3 and R4 with the nitrogen atom to which they are attached, defined above, being optionally substituted with one or more radicals chosen from halogen atoms and hydroxyl, alkoxy, NH2, NHalk, N(alk)2 radicals and alkyl, CH2-heterocycloalkyl, CH2-phenyl, CO-phenyl and S-heteroaryl radicals, such that, in the latter radicals, the alkyl, heterocycloalkyl, phenyl and heteroaryl radicals are themselves optionally substituted with one or more radicals chosen from halogen atoms and hydroxyl, alkyl and alkoxy radicals containing from 1 to 4 carbon atoms, NH2, NHalk and N(alk)2;
the said products of formula (I) being in any possible racemic, enantiomeric or diastereoisomeric isomer form, and also the addition salts with mineral and organic acids or with mineral and organic bases of the said products of formula (I).

One subject of the present invention is thus the products of formula (I) as defined above, in which [symbol] , X, A and W have the meanings given hereinabove or hereinbelow, Ra represents a hydrogen atom or a chlorine atom, or the radical:

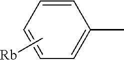

with Rb representing a halogen atom or a radical S-heteroaryl optionally substituted with a radical chosen from halogen atoms and hydroxyl, alkyl and alkoxy radicals containing from 1 to 4 carbon atoms, NH2, NHalk and N(alk)2, the said products of formula (I) being in any possible racemic, enantiomeric or diastereoisomeric isomer form, and also the addition salts with mineral and organic acids or with mineral and organic bases of the said products of formula (I).

As regards the cyclic radicals that may be formed by R1 and R2 or R3 and R4 with the nitrogen atom to which they are attached, these radicals optionally containing one or more other heteroatoms chosen from O, S, N and NH, with the optional S possibly being in the form SO or SO2; these radicals, including the optional NH they contain, may thus be optionally substituted especially with a radical chosen from alkyl, alkoxy, cycloalkyl and heterocycloalkyl, which are themselves optionally substituted with one or more radicals chosen from halogen atoms and alkyl, alkoxy, NH2, NHalk or N(alk)2 radicals.

In the products of formula (I) and in the text hereinbelow:

the term alkyl radical (or Alk) denotes linear and, where appropriate, branched methyl, ethyl, propyl, isopropyl, butyl, isobutyl, sec-butyl, tert-butyl, pentyl, isopentyl, hexyl, isohexyl and also heptyl, octyl, nonyl and decyl radicals, and also the linear or branched positional isomers thereof: alkyl radicals containing from 1 to 6 carbon atoms and more particularly alkyl radicals containing from 1 to 4 carbon atoms of the above list are preferred;
the term alkoxy radical denotes linear and, where appropriate, branched methoxy, ethoxy, propoxy or isopropoxy, secondary or tertiary linear butoxy, pentoxy or hexoxy, and also the linear or branched positional isomers thereof: alkoxy radicals containing from 1 to 4 carbon atoms of the above list are preferred;
the term halogen atom denotes a chlorine, bromine, iodine or fluorine atom and preferably the chlorine, bromine or fluorine atom.

the term cycloalkyl radical denotes a saturated carbocyclic radical containing 3 to 10 carbon atoms and thus especially denotes cyclopropyl, cyclobutyl, cyclopentyl and cyclohexyl radicals and most particularly cyclopropyl, cyclopentyl and cyclohexyl radicals;

the term heterocycloalkyl radical thus denotes a 3- to 10-membered monocyclic or bicyclic carbocyclic radical interrupted with one or more heteroatoms, which may be identical or different, chosen from oxygen, nitrogen and sulfur atoms: examples that may be mentioned include morpholinyl, thiomorpholinyl, aziridyl, azetidyl, piperazinyl, piperidyl, homopiperazinyl, pyrrolidinyl, imidazolidinyl, pyrazolidinyl, tetrahydrofuryl, tetrahydrothienyl, tetrahydropyranyl and oxodihydropyridazinyl radicals, or alternatively oxetanyl or thietanyl radicals, all these radicals being optionally substituted; it may be noted that these heterocycloalkyl radicals may comprise a bridge formed from two ring members to form, for example, an oxa-5-azabicyclo[2.2.1]heptane or an azaspiro[3,3]heptane radical or other azabicycloalkane or azaspiroalkane rings.

the terms aryl and heteroaryl denote unsaturated or partially unsaturated monocyclic or bicyclic, carbocyclic and heterocyclic radicals, respectively, which are not more than 12-membered, possibly containing a —C(O) ring member, the heterocyclic radicals containing one or more heteroatoms, which may be identical or different, chosen from O, N and S with N, where appropriate, being optionally substituted;

the term aryl radical thus denotes 6- to 12-membered monocyclic or bicyclic radicals, for instance phenyl, naphthyl, biphenyl, indenyl, fluorenyl and anthracenyl radicals, more particularly phenyl and naphthyl radicals and even more particularly the phenyl radical. It may be noted that a carbocyclic radical containing a —C(O) ring member is, for example, the tetralone radical;

the term heteroaryl radical thus denotes 5- to 12-membered monocyclic or bicyclic radicals; monocyclic heteroaryl radicals, for instance thienyl radicals such as 2-thienyl and 3-thienyl, furyl such as 2-furyl, 3-furyl, pyranyl, pyrrolyl, pyrrolinyl, pyrazolinyl, imidazolyl, pyrazolyl, pyridyl such as 2-pyridyl, 3-pyridyl and 4-pyridyl, pyrazinyl, pyrimidinyl, pyridazinyl, oxazolyl, thiazolyl, isothiazolyl, diazolyl, thiadiazolyl, thiatriazolyl, oxadiazolyl, isoxazolyl such as 3- or 4-isoxazolyl, furazanyl, free or salified tetrazolyl, all these radicals being optionally substituted, among which more particularly are thienyl radicals such as 2-thienyl and 3-thienyl, furyl such as 2-furyl, pyrrolyl, pyrrolinyl, pyrazolinyl, imidazolyl, pyrazolyl, oxazolyl, isoxazolyl, pyridyl and pyridazinyl, these radicals being optionally substituted; bicyclic heteroaryl radicals, for instance benzothienyl radicals such as 3-benzothienyl, benzothiazolyl, quinolyl, isoquinolyl, dihydroquinolyl, quinolone, tetralone, adamantyl, benzofuryl, isobenzofuryl, dihydrobenzofuryl, ethylenedioxyphenyl, thianthrenyl, benzopyrrolyl, benzimidazolyl, benzoxazolyi, thionaphthyl, indolyl, azaindolyl, indazolyl, purinyl, thienopyrazolyl, tetrahydroindazolyl, tetrahydrocyclopentapyrazolyl, dihydrofuropyrazolyl, tetrahydropyrrolopyrazolyl, oxotetrahydropyrrolopyrazolyl, tetrahydropyranopyrazolyl, tetrahydropyridinopyrazolyl or oxodihydropyridinopyrazolyl, all these radicals being optionally substituted.

As examples of heteroaryl or bicyclic radicals, mention may be made more particularly of pyrimidinyl, pyridyl, pyrrolyl, azaindolyl, indazolyl or pyrazolyl radicals, optionally substituted with one or more identical or different substituents as indicated above.

The carboxyl radical(s) of the products of formula (I) may be salified or esterified with various groups known to those skilled in the art, among which examples that may be mentioned include:

among the salification compounds, mineral bases such as, for example, one equivalent of sodium, potassium, lithium, calcium, magnesium or ammonium, or organic bases, for instance methylamine, propylamine, trimethylamine, diethylamine, triethylamine, N,N-dimethylethanolamine, tris(hydroxymethyl)-aminomethane, ethanolamine, pyridine, picoline, dicyclohexylamine, morpholine, benzylamine, procaine, lysine, arginine, histidine or N-methyl-glucamine,

among the esterification compounds, alkyl radicals to form alkoxycarbonyl groups, for instance methoxycarbonyl, ethoxycarbonyl, tert-butoxycarbonyl or benzyloxycarbonyl, these alkyl radicals possibly being substituted with radicals chosen, for example, from halogen atoms and hydroxyl, alkoxy, acyl, acyloxy, alkylthio, amino and aryl radicals, for instance in chloromethyl, hydroxypropyl, methoxymethyl, propionyloxymethyl, methylthiomethyl, dimethylaminoethyl, benzyl or phenethyl groups.

The addition salts with mineral or organic acids of the products of formula (I) may be, for example, the salts formed with hydrochloric, hydrobromic, hydriodic, nitric, sulfuric, phosphoric, propionic, acetic, trifluoroacetic, formic, benzoic, maleic, fumaric, succinic, tartaric, citric, oxalic, glyoxylic, aspartic or ascorbic acid, alkylmonosulfonic acids, for instance methanesulfonic acid, ethanesulfonic acid, propanesulfonic acid, alkyldisulfonic acids, for instance methanedisulfonic acid, α,β-ethanedisulfonic acid, arylmonosulfonic acids such as benzenesulfonic acid, and aryldisulfonic acids.

It may be recalled that stereoisomerism may be defined in its broadest sense as isomerism of compounds having the same structural formulae, but whose various groups are arranged differently in space, especially such as in monosubstituted cyclohexanes in which the substituent may be in an axial or equatorial position, and the various possible rotational conformations of ethane derivatives. However, another type of stereoisomerism exists, due to the various spatial arrangements of fixed substituents, either on double bonds, or on rings, which is often referred to as geometrical isomerism or cistrans isomerism. The term stereoisomers is used in the present patent application in its broadest sense and thus concerns all the compounds indicated above.

The cyclic radicals that may be formed, on the one hand, by R1 and R2 with the nitrogen atom to which they are attached, and, on the other hand, by R3 and R4 with the nitrogen atom to which they are attached, are optionally substituted with one or more radicals chosen from those indicated above for the possible substituents on the heterocycloalkyl radicals, i.e. one or more radicals chosen from halogen atoms and the following radicals: hydroxyl, oxo, alkoxy, NH2; NHalk, N(alk)2, and alkyl, heterocycloalkyl, CH2-heterocycloalkyl, phenyl, CH2-phenyl, heteroaryl and CO-phenyl radicals, such that in the latter radicals the alkyl, heterocycloalkyl and phenyl radicals are themselves optionally substituted with one or more radicals chosen from halogen atoms and the following radicals: hydroxyl, oxo, alkyl and alkoxy containing from 1 to 4 carbon atoms, NH2; NHalk and N(alk)2.

The cyclic radicals that may be formed, on the one hand, by R1 and R2 with the nitrogen atom to which they attached, and, on the other hand, by R3 and R4 with the nitrogen atom to which they are attached, are especially optionally substituted with one or more identical or different radicals chosen from halogen atoms and alkyl, hydroxyl, alkoxy, CH2-pyrrolidinyl, CH2-phenyl, heteroaryl and phenyl radicals, in which the alkyl, pyrrolidinyl and phenyl radicals are themselves optionally substituted with one or more identical or different radicals chosen from halogen atoms and alkyl, hydroxyl, oxo and alkoxy radicals.

The heterocycloalkyl radicals as defined above especially represent azepanyl, morpholinyl, pyrrolidinyl, piperidyl and piperazinyl radicals, which are themselves optionally substituted, as defined hereinabove or hereinbelow.

When NR1R2 or NR3R4 forms a ring as defined above, such an amine ring may be chosen especially from pyrrolidinyl, pyrazolidinyl, pyrazolinyl, piperidyl, azepinyl, morpholino and piperazinyl radicals, these radicals themselves being optionally substituted as indicated hereinabove or hereinbelow: for example with one or more radicals, which may be identical or different, chosen from halogen atoms and alkyl, hydroxyl, alkoxy, phenyl and CH2-phenyl radicals, the alkyl or phenyl radicals themselves being optionally substituted with one or more identical or different radicals chosen from halogen atoms and alkyl, hydroxyl and alkoxy radicals.

The ring NR1R2 or NR3R4 may be chosen more particularly from pyrrolidinyl and morpholino radicals optionally substituted with one or two alkyl or piperazinyl radicals optionally substituted on the second nitrogen atom with an alkyl, phenyl or CH2-phenyl radical, which are themselves optionally substituted with one or more identical or different radicals chosen from halogen atoms and alkyl, hydroxyl and alkoxy radicals.

A subject of the present invention is especially the products of formula (I) in which A represents NH, the substituents Ra, X and W being chosen from all the values defined for these radicals hereinabove or hereinbelow, the said products of formula (I) being in any possible racemic, enantiomeric or diastereoisomeric isomer form, and also the addition salts with mineral and organic acids or with mineral and organic bases of the said products of formula (I).

A subject of the present invention is especially the products of formula (I) in which A represents S, the substituents Ra, X and W being chosen from all the values defined for these radicals hereinabove or hereinbelow, the said products of formula (I) being in any possible racemic, enantiomeric or diastereoisomeric isomer form, and also the addition salts with mineral and organic acids or with mineral and organic bases of the said products of formula (I).

In particular, the present invention relates to the products of formula (I) corresponding to formula (Ia) or (Ib):

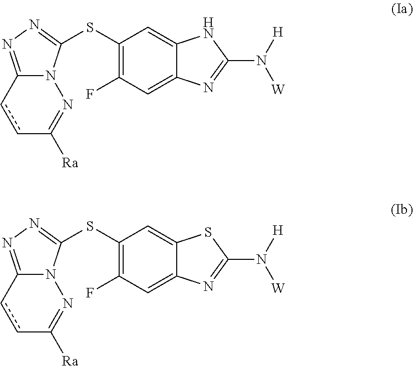

in which [symbol] , Ra and W are chosen from all the meanings indicated hereinabove or hereinbelow,
the said products of formula (Ia) and (Ib) being in any possible racemic, enantiomeric or diastereoisomeric isomer form, and also the addition salts with mineral and organic acids or with mineral and organic bases of the said products of formulae (Ia) and (lb).

The present invention thus particularly relates to the products of formula (I) as defined hereinabove or hereinbelow, in which [symbol] represents a single bond, corresponding to the products of formula (I′):

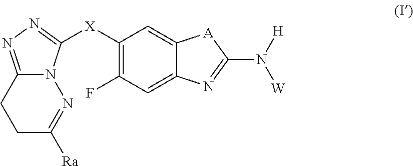

the substituents Ra, X, A and W are chosen from all the meanings indicated hereinabove or hereinbelow,

the said products of formula (I′) being in any possible racemic, enantiomeric or diastereoisomeric isomer form, and also the addition salts with mineral and organic acids or with mineral and organic bases of the said products of formula (I′).

The present invention thus particularly relates to the products of formula (I) as defined hereinabove or hereinbelow, in which [symbol] represents a double bond, corresponding to the products of formula (I″):

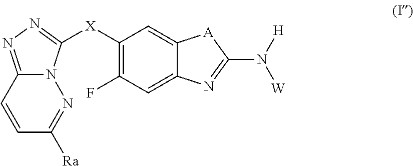

in which the substituents Ra, X, A and W are chosen from all the meanings indicated hereinabove or hereinbelow,
the said products of formula (I″) being in any possible racemic, enantiomeric or diastereoisomeric isomer form, and also the addition salts with mineral and organic acids or with mineral and organic bases of the said products of formula (I″).

The present invention thus particularly relates to the products of formula (Ia) as defined hereinabove or hereinbelow, in which [symbol] represents a single bond, corresponding to the products of formula (Ia′):

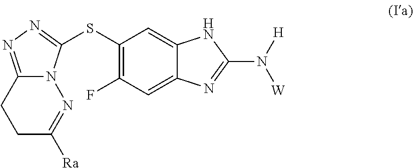

in which Ra and W are chosen from all the meanings indicated hereinabove or hereinbelow,
the said products of formula (I′a) being in any possible racemic, enantiomeric or diastereoisomeric isomer form, and also the addition salts with mineral and organic acids or with mineral and organic bases of the said products of formula (I′a),

The present invention thus particularly relates to the products of formula (Ia) as defined hereinabove or hereinbelow, in which [symbol] represents a double bond, corresponding to the products of formula (I″a):

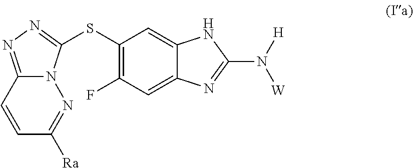

in which Ra and W are chosen from all the meanings indicated hereinabove or hereinbelow,
the said products of formula (I″a) being in any possible racemic, enantiomeric or diastereoisomeric isomer form, and also the addition salts with mineral and organic acids or with mineral and organic bases of the said products of formula (I′a).

The present invention thus particularly relates to the products of formula (Ib) as defined hereinabove or hereinbelow, in which [symbol] represents a single bond, corresponding to the products of formula (I′b):

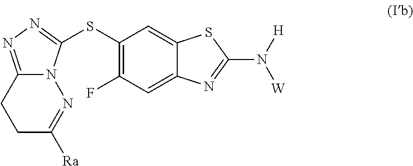

in which Ra and W are chosen from all the meanings indicated hereinabove or hereinbelow,
the said products of formula (I′b) being in any possible racemic, enantiomeric or diastereoisomeric isomer form, and also the addition salts with mineral and organic acids or with mineral and organic bases of the said products of formula (I′b).

The present invention thus particularly relates to the products of formula (Ib) as defined hereinabove or hereinbelow, in which [symbol] represents a double bond, corresponding to the products of formula (I″b):

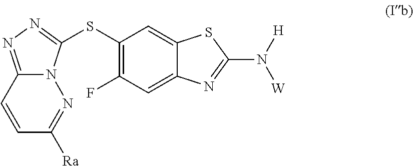

in which Ra and W are chosen from all the meanings indicated hereinabove or hereinbelow,
the said products of formula (I″b) being in any possible racemic, enantiomeric or diastereoisomeric isomer form, and also the addition salts with mineral and organic acids or with mineral and organic bases of the said products of formula (I″b).

When, in the products of formula (I). Ra represents the radical:

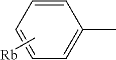

Rb is especially in the para position.

When Rb defined above represents a halogen atom, Rb especially represents fluorine.

A subject of the present invention is most particularly the products of formula (I) as defined above, corresponding to the following formulae:
1-(6-{[6-(cyclohexyloxy)[1,2,4]triazolo[4,3-b]pyridazin-3-yl]sulfanyl}-5-fluoro-1,3-benzothiazol-2-yl)-3-[2-(morpholin-4-yl)ethyl]urea
2-methylpropan-2-yl(5-fluoro-6-{[6-(4-fluorophenyl)[1,2,4]triazolo[4,3-b]-pyridazin-3-yl]sulfanyl}-1,3-benzothiazol-2-yl)carbamate
5-fluoro-6-{[6-(4-fluorophenyl)[1,2,4]triazolo[4,3-b]pyridazin-3-yl]sulfanyl}-1,3-benzothiazol-2-amine
1-(5-fluoro-6-{[6-(4-fluorophenyl)[1,2,4]triazolo[4,3-b]pyridazin-3-yl]sulfanyl}-1,3-benzothiazol-2-yl)-3-[2-(morpholin-4-yl)ethyl]urea
1-(6-{[6-(cyclopropylamino)[1,2,4]triazolo[4,3-b]pyridazin-3-yl]sulfanyl}-5-fluoro-1,3-benzothiazol-2-yl)-3-[2-(morpholin-4-yl)ethyl]urea
1-(6-{[6-(cyclohexylamino)[1,2,4]triazolo[4,3-b]pyridazin-3-yl]sulfanyl}-5-fluoro-1,3-benzothiazol-2-yl)-3-[2-(morpholin-4-yl)ethyl]urea
2-(4-cyclopropylpiperazin-1-yl)-N-{6-[(6-ethoxy[1,2,4]triazolo[4,3-b]pyridazin-3-yl)sulfanyl]-5-fluoro-1,3-benzothiazol-2-yl}acetamide
N-(6-{[6-(cyclobutyloxy)[1,2,4]triazolo[4,3-b]pyridazin-3-yl]sulfanyl}-5-fluoro-1,3-benzothiazol-2-yl)-2-(4-cyclopropylpiperazin-1-yl)acetamide
N-{6-[(6-ethoxy[1,2,4]triazolo[4,3-b]pyridazin-3-yl)sulfanyl]-5-fluoro-1,3-benzothiazol-2-yl}-2-(4-ethylpiperazin-1-yl)acetamide
N-(6-{[6-(cyclobutyloxy)[1,2,4]triazolo[4,3-b]pyridazin-3-yl]sulfanyl}-5-fluoro-1,3-benzothiazol-2-yl)-2-(4-ethylpiperazin-1-yl)acetamide
2-(4-cyclopropylpiperazin-1-yl)-N-(5-fluoro-6-{[6-(oxetan-3-yloxy)[1,2,4]triazolo[4,3-b]pyridazin-3-yl]sulfanyl}-1,3-benzothiazol-2-yl)acetamide
2-(4-cyclopropylpiperazin-1-yl)-N-(5-fluoro-6-{[6-(tetrahydrofuran-3-yloxy)[1,2,4]triazolo[4,3-b]pyridazin-3-yl]sulfanyl}-1,3-benzothiazol-2-yl)acetamide
   and also the addition salts with mineral and organic acids or with mineral and organic bases of the said products of formula (I).

A subject of the present invention is also any process for preparing the products of formula (I) as defined above.

A subject of the present invention is thus any process for preparing the products of formula (I) as defined above, in which A represents NH,

A subject of the present invention is thus any process for preparing the products of formula (I) as defined above, in which A represents S.

The products according to the invention may be prepared from conventional methods of organic chemistry. Schemes 1, 2, 2 bis, 3, 4, 5 and 6 hereinbelow illustrate methods used for the preparation of the products of formula (I). In this respect, they shall not constitute a limitation of the scope of the invention, as regards the methods for preparing the claimed compounds.

The products of formula (I) as defined above according to the present invention may thus especially be prepared according to the processes described in Schemes 1, 2, 2bis, 3, 4, 5 and 6 hereinbelow.

A subject of the present invention is thus also the process for preparing products of formula (I) according to Scheme 1 as defined below.

A subject of the present invention is thus also the process for preparing products of formula (I) according to Scheme 2 as defined below.

A subject of the present invention is thus also the process for preparing products of formula (I) according to Scheme 2bis as defined below.

A subject of the present invention is thus also the process for preparing products of formula (I) according to Scheme 3 as defined below.

A subject of the present invention is thus also the process for preparing products of formula (I) according to Scheme 4 as defined below.

A subject of the present invention is thus also the process for preparing products of formula (I) according to Scheme 5 as defined below.

A subject of the present invention is thus also the process for preparing products of formula (I) according to Scheme 6 as defined below.

Similarly, among the products of formula (I) as defined above in which [symbol] represents a single or double bond, the products of formula (I′) are defined, which represent products of formula (I) in which [symbol] represents a single bond and products of formula (I″) which represent products of formula (I) in which [symbol] represents a double bond,

and similarly, for the synthetic intermediates as defined below of formulae (a), (b), (c), (d), (e) and (f) in which [symbol] represents a single or double bond, the compounds of formulae (a′), (b′), (c′), (d′), (e′) and (f′) are defined, in which [symbol] represents a single bond, and the compounds of formulae (a″), (b″), (c″), (d″), (e″) and (f″) in which [symbol] represents a double bond.

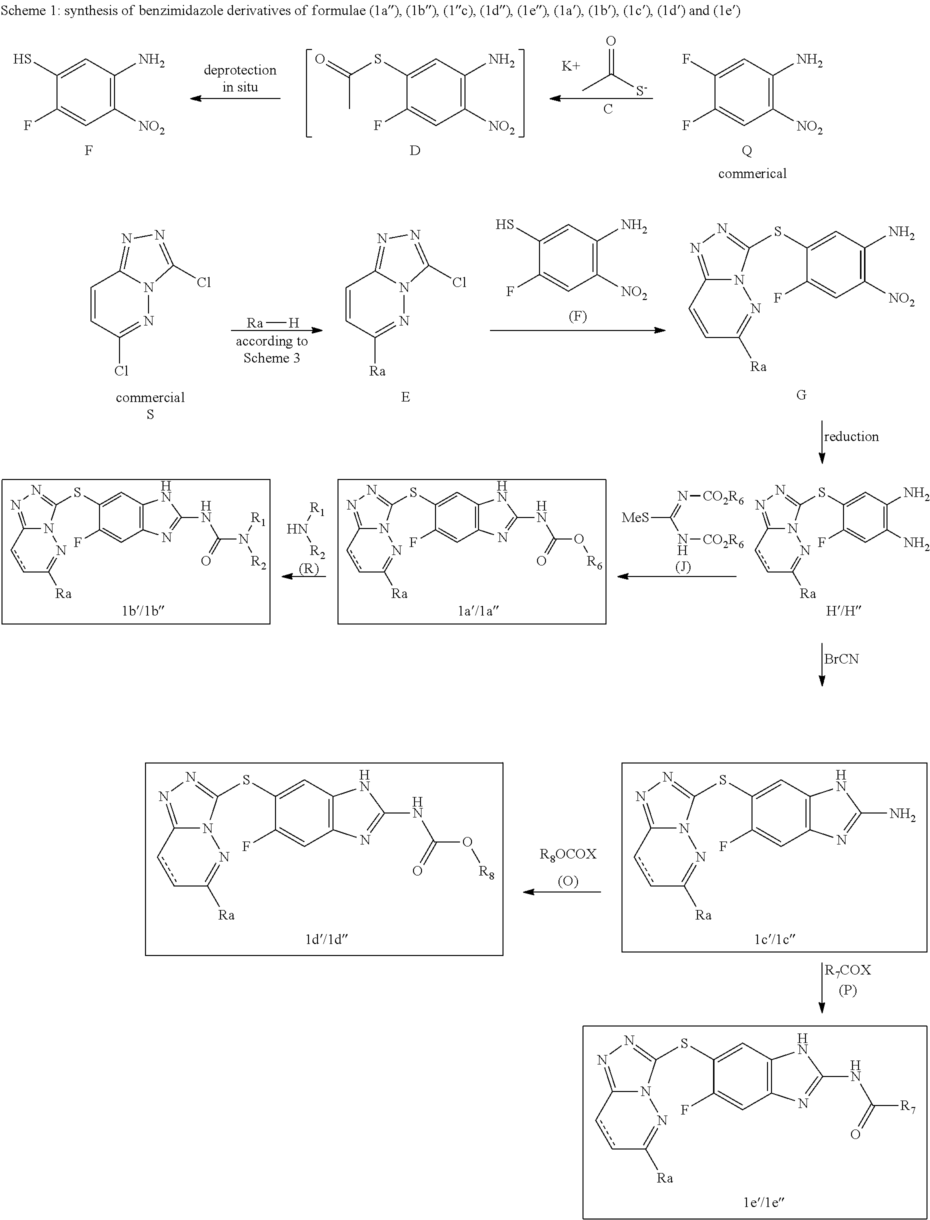

In Scheme 1 above, the substituent Ra has the meanings given above for the products of formulae (I′) and (I″) and the groups CONR1R2, COR6 and COR7, which constitute W, may take the values of W as defined above for the products of formulae (I′) and (I″), when W≠H

In the above Scheme 1, the benzimidazoles of general formulae (1a″), (1b″), (1c″), (1d″) and (1e″) and also the reduced analogues thereof of general formulae (1a′), (1b′), (1c′), (1d′) and (1e′) may be prepared from commercial 3,6-dichloro[1,2,4]triazolo[4,3-b]pyridazine of formula (S).

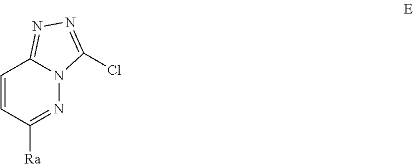

The compounds (E) may be obtained by the routes described in Scheme 3 below

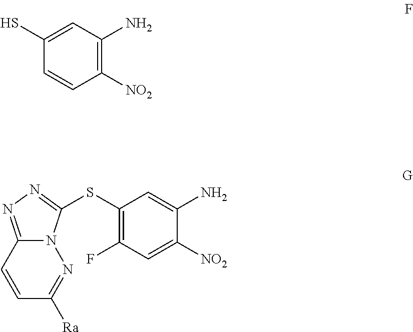

The compounds (G) may be obtained, for example, by reacting 2-fluoro-4-nitro-5-aminobenzenethiol of formula (F) with the compounds of formula (E). The compound of formula (F) may be obtained by introducing the thiol function onto the derivative 2-nitro-4,5-difluoroaniline (Q) (commercial compound), for example, in the presence of potassium thioacetate in a solvent such as N,N-dimethylformamide, at a temperature in the region of 20° C.

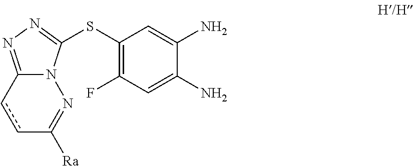

The compounds (H″) such that [symbol] represent a double bond may be obtained, for example, via reduction with iron (0) of the compounds of formula (G), in a solvent such as methanol, in the presence of acetic acid, at a temperature in the region of 70° C.

The compounds (H′) such that [symbol] represent a single bond may be obtained, for example, via reduction with zinc (0) of the compounds of formula (G), in the presence of acetic acid, at a temperature in the region of 20° C.

More particularly, the carbamates of general formulae (1a′) and (1a″) may especially be prepared as described in patent WO 03/028721 A2, but starting, respectively, with a 2-fluoro-4,5-diaminophenyl sulfide of formulae (H′) and (H″) and with a pseudo thiourea of formula (J), in the presence of acetic acid and in a protic solvent such as methanol, at a temperature in the region of 80° C.

More particularly, the benzimidazoles of general formulae (1 b′) and (1b″) may be prepared, respectively, by reaction of an amine NHR1R2 of formula (R) (with R1 and R2 as defined above) with a carbamate of formulae (1a′) and (1a″), for example in the presence of an aprotic solvent such as 1-methyl-2-pyrrolidinone. The reaction is performed, for example, at a temperature in the region of 120° C., in a sealed tube under microwaves.

More particularly, the 2-aminobenzimidazoles of general formulae (1c′) and (1c″) may be prepared, for example, by reacting cyanogen bromide with a compound of formulae (H′) and (H″), respectively, in the presence of a protic solvent such as ethanol. The reaction is performed at a temperature in the region of 80° C.

More particularly, the carbamates of general formulae (1d′) and (1d″) may be obtained by reacting a chlorocarbonate of formula (O) (X═Cl) with a compound of general formulae (1c′) and (1c″), for example in a solvent such as tetrahydrofuran, in the presence of a base such as sodium hydrogen carbonate, at a temperature in the region of 20° C.

More particularly, the carboxamides (1e′) and (1e″) may be obtained, respectively, from the amines of general formulae (Ic′) and (1c″)

by reacting the amines (1c′) and (1c′) with an acid chloride of formula (P) (X═Cl), in the presence, for example, of a solvent such as pyridine, at a temperature in the region of 20° C.;

by reacting the amines (1c′) and (1c′) with an acid anhydride of formula (P) (X═OCOR7), in the presence, for example, of solvent such as pyridine at a temperature in the region of 20° C.;
by coupling the amines (1c′) and (1c′) with an acid of formula (P) (X═OH) under the conditions described, for example, by D. D. DesMarteau; V. Montanari (Chem. Lett., 2000 (9). 1052), in the presence of 1-hydroxybenzotriazole and 1-(3-dimethylaminopropyl)-3-ethylcarbodiimide and in the presence of a base such as triethylamine, at a temperature in the region of 40° C.

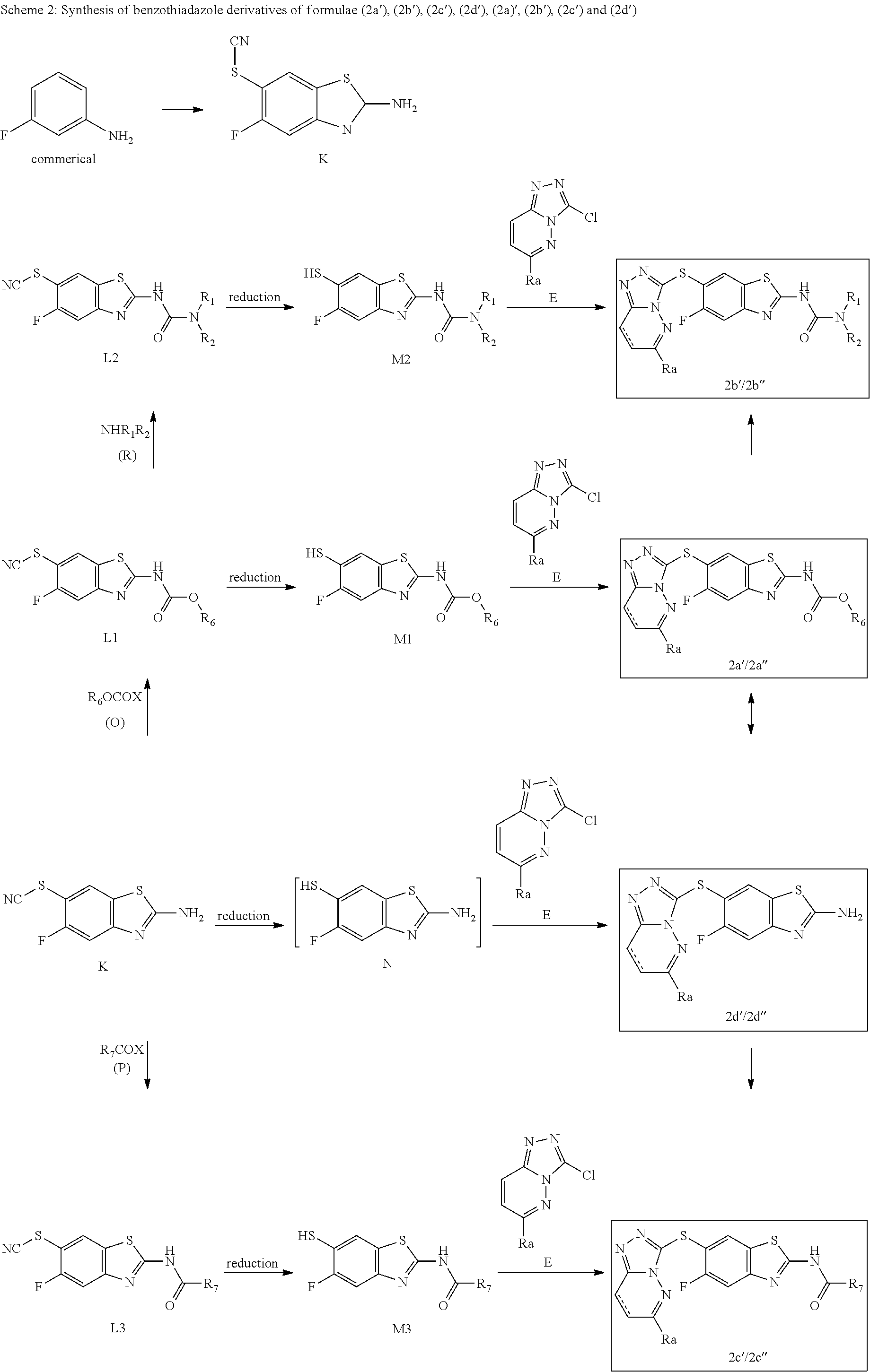

In Scheme 2 above, the substituent Ra has the meanings indicated above for the products of formulae (I′) and (I″) and the groups CONR1R2, COR6 and COR7, which constitute W, may take values of W as defined above for the products of formulae (I′) and (I″), when W≠H.

In Scheme 2 above, the benzothiazoles of general formulae (2a″), (2b″), (2c″) and (2d″) and the reduced analogues thereof of general formulae (2a′), (2b′), (2c′) and (2d′) may be prepared from 2-amino-5-fluoro-1,3-benzothiazol-6-yl thiocyanate (K).

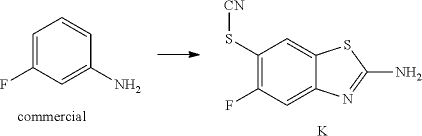

In Scheme 2 above, 2-amino-5-fluoro-1,3-benzothiazol-6-yl thiocyanate (K) may be prepared from 3-fluoroaniline in the manner described by K. Papke and R. Pohioudek-Fabini in Pharmazie; GE; 22, 5 1967, P229-233

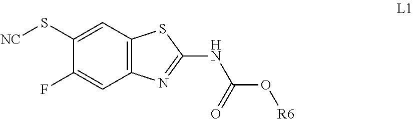

The carbamates of general formula (L1) may be obtained, for example, by reacting a chlorocarbonate of formula (O) (X═Cl) with 2-amino-5-fluoro-1,3-benzothiazol-6-yl thiocyanate (K), in a solvent such as tetrahydrofuran, in the presence of a base such as sodium hydrogen carbonate, at a temperature in the region of 20° C.

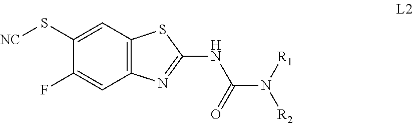

The compounds of general formula (L2) may be obtained, for example, by reacting the carbamates of formula (L1) in which R6=phenyl with amines NHR1R2 of formula (R) (with R1 and R2 as defined above), in the presence of an aprotic solvent such as tetrahydrofuran, at a temperature in the region of 20° C.

The ureas (2b′) and (2b″) may be obtained, for example, respectively, from the carbamates (2a′) and (2a″) in which R6=phenyl, in the same manner as the ureas (L2) are obtained by reacting amines on the carbamates of the type (L1).

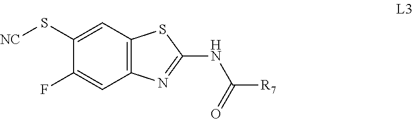

The compounds of general formula (L3) may be obtained, for example:

by reacting an acid chloride of formula (P) (X═Cl) with 2-amino-5-fluoro-1,3-benzothiazol-6-yl thiocyanate (K), in the presence, for example, of a solvent such as pyridine, at a temperature in the region of 20° C.,

by reacting an acid anhydride of formula (P) (X═OCOR7) with 2-amino-5-fluoro-1,3-benzothiazol-6-yl thiocyanate (K), in the presence, for example, of a solvent such as pyridine, at a temperature in the region of 20° C.,

by coupling 2-amino-5-fluoro-1,3-benzothiazol-6-yl thiocyanate (K) with an acid of formula (P) (X═OH) under the conditions described, for example, by D. D. DesMarteau; V. Montanan i (Chem. Lett, 2000 (9). 1052), in the presence of 1-hydroxybenzotriazole and 1-(3-dimethylaminopropyl)-3-ethylcarbodiimide and in the presence of a base such as triethylamine, at a temperature in the region of 40° C.

In the same manner that the carboxamides (L3) may be obtained via acylation of the amine (K), the carboxamides (2c′) and (2c″) may be obtained, respectively, from the amines (2d′) and (2d″).

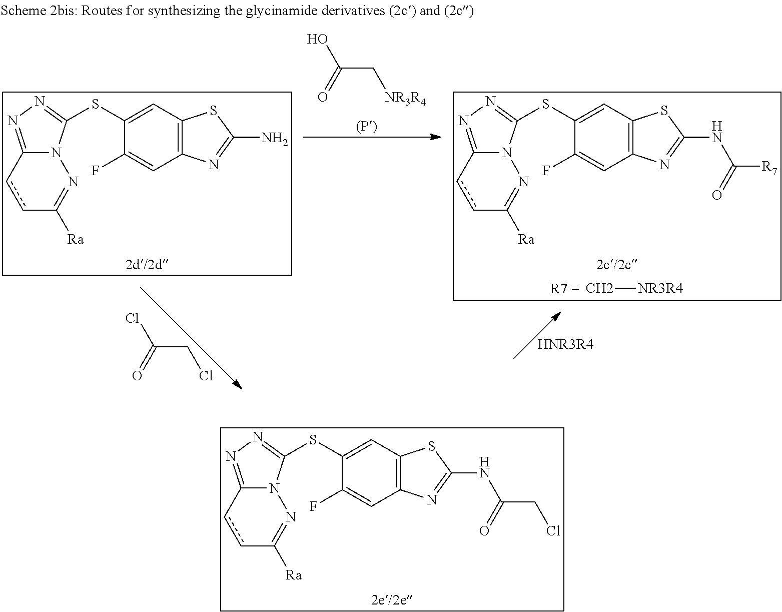

In Scheme 2bis above, the substituent R7 may take the meaning of an aminomethyl group. These glycinamides (2c′/2c″) may be obtained by coupling the amines (2d′) and (2d″) with a glycidic acid (P′) using the methods described above for the acids (P) (X═OH).

The glycidic acids (P′) may be prepared from bromoacetic acid and amines HNR3R4 under conditions similar to those described by D. T. Witiak et al.; J. Med. Chem. 1985, 28, 1228.

Alternatively, the amines (2d′) and (2d″) may be treated with fluoroacetyl chloride in the presence of a base such as pyridine, triethylamine or N-methylmorpholine, in a solvent such as dichloromethane at a temperature in the region of 0° C. to 20° C. The a-chloroacetamides (2e′/2e″) thus formed can react with amines of the type HNR3R4, as defined above, in a solvent such as pyridine at a temperature in the region of 20° C., to give the derivatives (2c′/2c″) as defined in Scheme 2bis above.

The compounds of general formulae (M1), (M2) and (M3) may be obtained, for example, by reduction of compounds of general formula (L1), (L2) or (L3) with DL-dithiothreitol, in the presence of sodium hydrogen carbonate, in a solvent such as ethanol and at a temperature in the region of 80° C.

The compound of general formula (N) may be prepared in situ by reduction of the compound of formula (K), for example with sodium borohydride in a solvent such as N,N-dimethylformamide, in the presence of a base such as triethylamine and at a temperature in the region of 95° C. or between 20° C. and 95° C., or, alternatively, for example, with DL-dithiotreitol, in the presence of sodium hydrogen carbonate, in a solvent such as ethanol, and at a temperature in the region of 80° C.

More particularly, the benzothiazoles of general formulae (2d′) and (2d″) may also be prepared, respectively, from carbamates of formulae (2a′) and (2a″) in which R6=t-butyl, by reaction, for example, with trifluoroacetic acid in a solvent such as dichloromethane, at a temperature in the region of 20° C.

Reciprocally, the benzothiazoles of general formulae (2a′) and (2a″) may also be prepared from benzothiazoles of formulae (2d′) and (2d″), respectively, for example, by reaction with a chlorocarbonate of formula (O) (X═Cl), in a solvent such as tetrahydrofuran, in the presence of a base such as sodium hydrogen carbonate, at a temperature in the region of 20° C.

More particularly, the benzothiazoles of general formulae (2a″), (2b″), (2c″) and (2d″) and the reduced analogues thereof of general formulae (2a′), (2b′), (2c′) and (2d′) may be prepared, for example:
1) either by coupling a compound of formula (E) with derivatives (M1), (M2) and (M3) and (N) generated in situ by reduction of the derivatives (L1), (L2), (L3) and (K) with sodium borohydride, in a solvent such as N,N-dimethylformamide, and in the presence of a base such as triethylamine, at a temperature in the region of 95° C. or between 50° C. and 95° C.;
2) or by coupling the isolated derivatives (M1), (M2) and (M3) and a compound of formula (E), in the presence of sodium borohydride in a solvent such as N,N-dimethylformamide and in the presence of a base such as triethylamine, at a temperature in the region of 95° C.;
3) or by coupling the isolated derivatives (M1), (M2) and (M3) and a compound of formula (E) under the conditions described, for example, by U. Schopfer et al. (Tetrahedron, 2001, 57, 3069) in the presence of n-tributylphosphine, potassium tert-butoxide, tris(dibenzylideneacetone)dipalladium(0) and bis(2-diphenylphosphinophenyl)ether, in a solvent such as toluene at a temperature in the region of 110° C.;
4) or by coupling a compound of formula (E) with derivatives (M1), (M2) and (M3) and (N) generated in situ by reduction of the derivatives (L1), (L2), (L3) and (K) in the presence of DL-dithiothreitol and sodium hydrogen carbonate, in a solvent such as ethanol and at a temperature in the region of 80° C.

The reductive conditions 1) and 2) may give products of formulae (2a), (2b), (2c) and (2d) such that [symbol] represent a single or double bond, whereas conditions 3) and 4) give products of formulae (2a), (2b), (2c) and (2d) such that [symbol] represent a double bond.

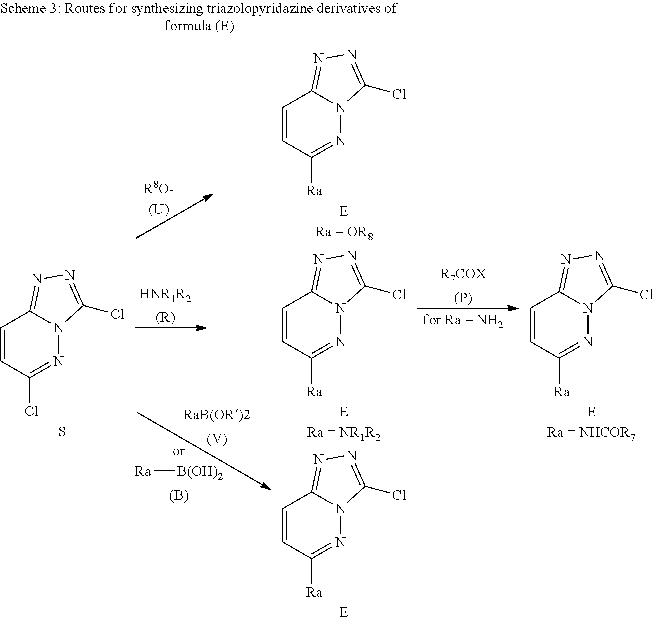

In Scheme 3 above, the substituents Ra, R1 and R2 have the meanings given hereinabove for the products of formulae (I′) and (I″). The substituent R7 represents an alkyl or cycloalkyl radical.

The substituent R8 represents:

either an alkyl radical optionally substituted with a chlorine atom, a hydroxyl radical or a heterocycloalkyl radical, which is itself optionally substituted,

or a cycloalkyl radical.

The compounds of formula (E) may be obtained, for example, as indicated in Scheme 3 above, from commercial 3,6-dichloro[1,2,4]triazolo[4,3-b]pyridazine of formula (S).

More particularly, the compounds of formula (E) in which Ra represents a radical OR8 may be obtained by treating 3,6-dichloro[1,2,4]triazolo[4,3-b]pyridazine (S) at a temperature in the region of 80° C. and in a solvent such as N,N-dimethylformamide with an alkoxide of formula (U), which is itself obtained by treating the corresponding alcohol with a base such as sodium hydride at a temperature in the region of 0° C. to 20° C.

More particularly, the compounds of formula (E) in which Ra represents a radical NR₁R₂ may be obtained by treating 3,6-dichloro[1,2,4]triazolo[4,3-b]-pyridazine (S) with an amine of formula (R), at a temperature in the region of 20° C. and in a solvent such as N,N-dimethylformamide, or, when NR1R2 is NH2, with aqueous ammonia, in a solvent such as dioxane, in a sealed tube, at a temperature between 70° C. and 90° C.

More particularly, the compounds of formula (E) in which Ra represents a radical NHCOR7 may be obtained by reacting a compound of general formula (E) with Ra═NH₂ with a compound of formula (P) as described for the compounds of general formulae (L3), (1e′) and (1e′).

More particularly, the compounds of formula (E) in which Ra represents an aryl or heteroaryl radical may be obtained, for example:
from the boronic acids of formula (B), in the presence of barium hydroxide octahydrate and (1,1′-bis(diphenylphosphino)ferrocene)dichloropalladium(II) in a solvent such as, for example, N,N-dimethylformamide, at a temperature in the region of 80° C.,
or alternatively, from the boronic esters of formula (V), in the presence of dichlorobis(triphenylphosphine)palladium in a solvent such as, for example, 1,2-dimethoxyethane, in the presence of a base such as 1N sodium hydroxide, at a temperature in the region of 80° C.

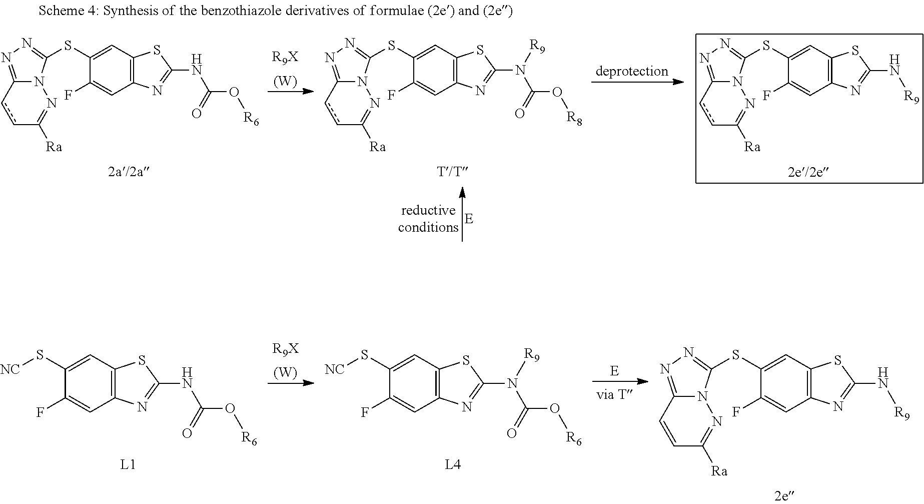

According to Scheme 4 above, the benzothiazoles of general formulae (2e′) and (2e″) may be prepared, respectively, from the compounds of formulae (2a′) and (2a″).

In Scheme 4 above, the substituent OR6 preferentially represents O-t-butyl. The substituent R9 represents an alkyl, cycloalkyl or heterocycloalkyl radical optionally substituted with an alkoxy or heterocycloalkyl radical or NR3R4 (R3 and R4 as defined above).

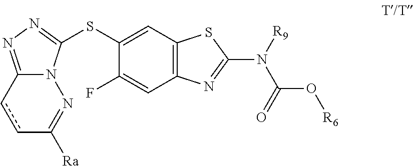

The carbamates of general formulae (T′) and (T″) may be obtained, respectively, by reacting carbamates of general formulae (2a′) and (2a″) with R6=tBu, preferentially, for example with alkyl halides of formula (W), in a solvent such as N,N-dimethylformamide, in the presence of sodium hydride, at a temperature of between 20 and 90° C.

The benzothiazoles of general formulae (2e′) and (2e″) may also be prepared from the compounds of formula (L1), with, preferably, R6=tBu, via the compounds of formulae (T′) and (T″).

More particularly, the compounds of general formulae (2e′) and (2e″) may be obtained, respectively, by treating the isolated compounds (T′) and (T″), for example, with trifluoroacetic acid, in a solvent such as dichloromethane, at a temperature in the region of 20° C.

Alternatively, the compounds of general formula (2e″) may be obtained directly by reacting the compounds of formulae (L4) and (E), via compound (T″) formed in situ, for example in the presence of DL-dithiothreitol and sodium hydrogen carbonate, in a solvent such as ethanol and at a temperature in the region of 80° C., optionally followed by an in situ treatment with trifluoroacetic acid at 20° C., if necessary.

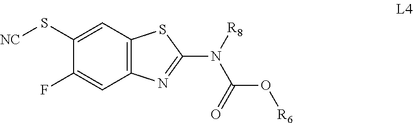

The carbamates of general formula (L4) may be obtained by reacting carbamates of general formula (L1), for example, with alkyl halides of formula (W), in a solvent such as N,N-dimethylformamide, in the presence of sodium hydride, at a temperature of between 20 and 90° C.

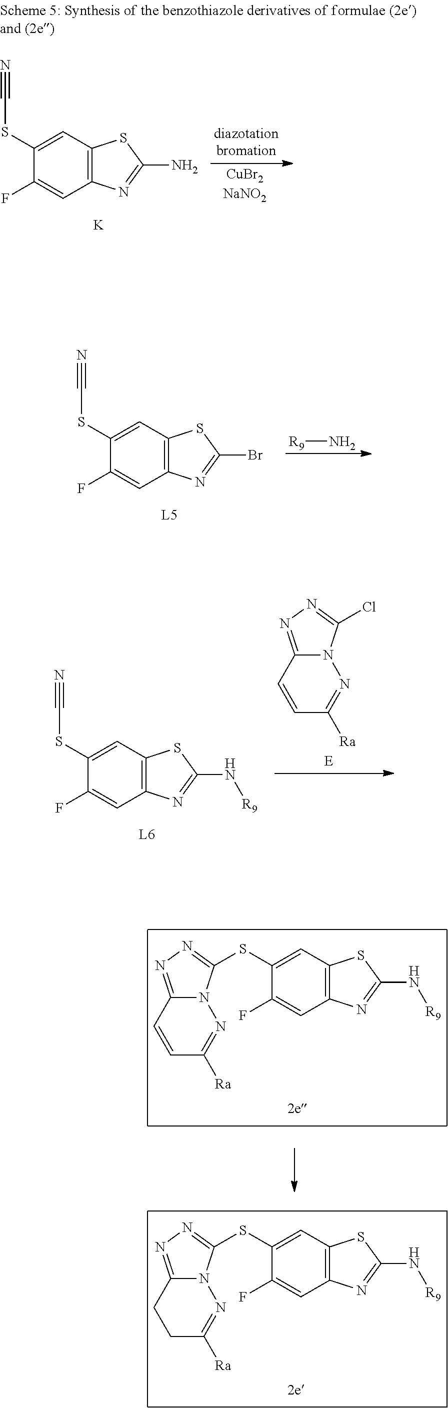

Alternatively, according to Scheme 5 above, the benzothiazoles of general formula (2e″) may be prepared from the compounds of formulae (L6) and (E), for example, in the presence of DL-dithiothreitol and sodium hydrogen carbonate, in a solvent such as ethanol and at a temperature in the region of 80° C.

The benzothiazoles of general formula (2e′) may be prepared from the compounds of formula (2e″) according to the methods described below for the preparation of the compounds (I′) from the compounds (I″).

The compounds of formula (L6) may be prepared from the 2-bromobenzothiazole derivative (L5) by treatment with a derivative NH2R9, for example, in a solvent such as tetrahydrofuran, at a temperature in the region of 20° C.

The substituent R9 represents an alkyl or cycloalkyl radical optionally substituted with an alkoxy or heterocycloalkyl radical or NR3R4 (R3 and R4 as defined above).

The compounds of formula (L5) may be prepared from 2-amino-5-fluoro-1,3-benzothiazol-6-yl thiocyanate (K) (commercial compound), for example, by treatment with an alkyl nitrite and cuprous bromide in a solvent such as acetonitrile, at a temperature in the region of 0-20° C., according to the method described by Jagabandhu Das et al. in J. Med. Chem. 2006, 49, 6819-6832.

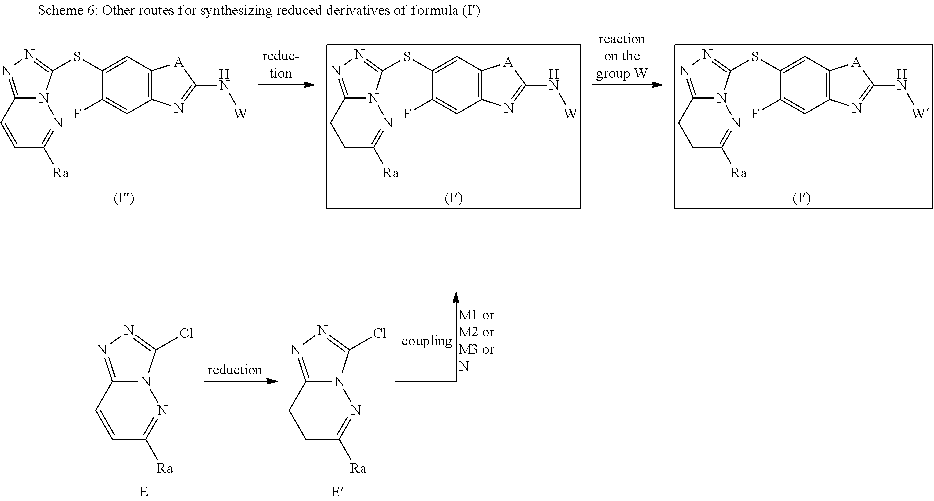

According to Scheme 6 above, the benzothiazoles of general formula (I′) may also be prepared from the compounds of formula (I″), via reduction, for example, with sodium borohydride, in a solvent such as ethanol, at a temperature in the region of 80° C., or via reduction with zinc (0) in the presence of acetic acid, at a temperature in the region of 20° C.

Alternatively, the compounds (I′) may also be prepared from the compounds of formula (E′) by coupling with compounds of the type M1, M2, M3 or N, obtained as intermediates via reduction of the compounds L1, L2, L3 or K in situ, as described above in Scheme 2. The compounds of the type M1, M2 or M3 may also be isolated and used for the coupling with (E′). The compounds (E′) may be obtained from the compounds of formula (E) by reduction, for example, with zinc (0) in the presence of acetic acid, at a temperature in the region of 20° C.

Alternatively, the compounds (I′) may also be prepared from other compounds (I′) via conversion of the group W into a group W′ of the same nature as defined above for W and according to the type of reaction defined in Scheme 2: conversion of 2d′/2d″ into 2a′/2a″ and into 2c′/2c″, conversion of 2a′/2a″ into 2d′/2d″ and into 2b′12b″.

In the compounds of general formula (I) as defined above, the sulfur S can be oxidized to sulfoxide SO or sulfone SO2 according to the methods known to those skilled in the art, if necessary protecting any reactive groups with suitable protecting groups.

Among the starting materials of formulae B, D, J, K, 0, P, P′, Q, R, S, U, V and W, some are known and may be obtained either commercially or according to the usual methods known to those skilled in the art, for example from commercial products.

It is understood by those skilled in the art that, to implement the processes according to the invention described previously, it may be necessary to introduce protecting groups for the amino, carboxyl and alcohol functions in order to avoid side reactions.

The following non-exhaustive list of examples of protection of reactive functions may be mentioned:

hydroxyl groups may be protected, for example, with alkyl radicals such as tert-butyl, trimethylsilyl, tert-butyldimethylsilyl, methoxymethyl, tetrahydropyranyl, benzyl or acetyl,

amino groups may be protected, for example, with acetyl, trityl, benzyl, tertbutoxycarbonyl, BOC, benzyloxycarbonyl or phthalimido radicals or other radicals known in peptide chemistry.

Acid functions may be protected, for example, in the form of esters formed with readily cleavable esters such as benzyl or tert-butyl esters or esters known in peptide chemistry.

A list of various protecting groups that may be used will be found in the manuals known to those skilled in the art and, for example, in patent BF 2 499 995.

It may be noted that intermediate products or products of formula (I) thus obtained via the processes indicated above may be subjected, if desired and if necessary, in order to obtain other intermediates or other products of formula (I), to one or more transformation reactions known to those skilled in the art, for instance:

a) a reaction for esterification of an acid function,
b) a reaction for saponification of an ester function to an acid function,
c) a reaction for reduction of the free or esterified carboxyl function to an alcohol function,
d) a reaction for conversion of an alkoxy function into a hydroxyl function, or alternatively of a hydroxyl function into an alkoxy function,
e) a reaction for removal of the protecting groups that may be borne by protected reactive functions,
f) a salification reaction with a mineral or organic acid or with a base to obtain the corresponding salt,
g) a reaction for resolution of racemic forms into resolved products, the said products of formula (I) thus obtained being in any possible racemic, enantiomeric or diastereoisomeric isomer form.

Reactions a) to g) may be performed under the usual conditions known to those skilled in the art, for instance those indicated hereinbelow.

a) The products described above may, if desired, undergo, on the possible carboxyl functions, esterification reactions that may be performed according to the usual methods known to those skilled in the art.
b) The possible conversions of ester functions into an acid function of the products described above may, if desired, be performed under the usual conditions known to those skilled in the art, especially by acidic or alkaline hydrolysis, for example with sodium hydroxide or potassium hydroxide in alcoholic medium, for instance in methanol, or alternatively with hydrochloric acid or sulfuric acid.

The saponification reaction may be performed according to the usual methods known to those skilled in the art, for instance in a solvent such as methanol, ethanol, dioxane or dimethoxyethane, in the presence of sodium hydroxide or potassium hydroxide.

c) The possible free or esterified carboxyl functions of the products described above may, if desired, be reduced to an alcohol function via the methods known to those skilled in the art: the possible esterified carboxyl functions may, if desired, be reduced to an alcohol function via the methods known to those skilled in the art and especially with lithium aluminium hydride in a solvent such as, for example, tetrahydrofuran, dioxane or ethyl ether.

The possible free carboxyl functions of the products described above may, if desired, be reduced to an alcohol function especially with boron hydride.

d) The possible alkoxy functions, especially such as methoxy, of the products described above may be, if desired, converted into a hydroxyl function under the usual conditions known to those skilled in the art, for example with boron tribromide in a solvent such as, for example, methylene chloride, with pyridine hydrobromide or hydrochloride, or alternatively with hydrobromic acid or hydrochloric acid in water or trifluoroacetic acid at reflux.
e) The removal of the protecting groups such as, for example, those indicated above may be performed under the usual conditions known to those skilled in the art, especially via acidic hydrolysis performed with an acid such as hydrochloric acid, benzenesulfonic acid or para-toluenesulfonic acid, formic acid or trifluoroacetic acid, or alternatively via catalytic hydrogenation.

The phthalimido group may be removed with hydrazine.

f) The products described above may, if desired, undergo salification reactions, for example with a mineral or organic acid or with a mineral or organic base according to the usual methods known to those skilled in the art: such a salification reaction may be performed, for example, in the presence of hydrochloric acid, for example, or tartaric acid, citric acid or methanesulfonic acid, in an alcohol, for instance ethanol or methanol.
g) The possible optically active forms of the products described above may be prepared by resolving racemic mixtures according to the usual methods known to those skilled in the art.

The products of formula (I) as defined above and the acid-addition salts thereof have advantageous pharmacological properties especially on account of their kinase-inhibiting properties as indicated above.

The products of the present invention are especially useful for treating tumours,

The products of the invention may thus also increase the therapeutic effects of commonly used antitumour agents.

These properties justify their therapeutic use, and a subject of the invention is particularly, as medicaments, the products of formula (I) as defined above, the said products of formula (I) being in any possible racemic, enantiomeric or diastereoisomeric isomer form, and also the addition salts with pharmaceutically acceptable mineral and organic acids or with pharmaceutically acceptable mineral and organic bases of the said products of formula (I).

A subject of the invention is most particularly, as medicaments, the products of formula (I) as defined above, corresponding to the following formulae:
1-(6-{[6-(cyclohexyloxy)[1,2,4]triazolo[4,3-b]pyridazin-3-yl]sulfanyl}-5-fluoro-1,3-benzothiazol-2-yl)-3-[2-(morpholin-4-yl)ethyl]urea
2-methylpropan-2-yl (5-fluoro-6-{[6-(4-fluorophenyl)[1,2,4]triazolo[4,3-b]pyridazin-3-yl]sulfanyl}-1,3-benzothiazol-2-yl)carbamate
5-fluoro-6-{[6-(4-fluorophenyl)[1,2,4]triazolo[4,3-b]pyridazin-3-yl]sulfanyl}-1,3-benzothiazol-2-amine
1-(5-fluoro-6-{[6-(4-fluorophenyl)[1,2,4]triazolo[4,3-b]pyridazin-3-yl]sulfanyl}-1,3-benzothiazol-2-yl)-3-[2-(morpholin-4-yl)ethyl]urea
1-(6-{[6-(cyclopropylamino)[1,2,4]triazolo[4,3-b]pyridazin-3-yl]sulfanyl}-5-fluoro-1,3-benzothiazol-2-yl)-3-[2-(morpholin-4-yl)ethyl]urea
1-(6-{[6-(cyclohexylamino)[1,2,4]triazolo[4,3-b]pyridazin-3-yl]sulfanyl}-5-fluoro-1,3-benzothiazol-2-yl)-3-[2-(morpholin-4-yl)ethyl]urea
2-(4-cyclopropylpiperazin-1-yl)-N-{6-[(6-ethoxy[1,2,4]triazolo[4,3-b]pyridazin-3-yl)sulfanyl]-5-fluoro-1,3-benzothiazol-2-yl}acetamide
N-(6-{[6-(cyclobutyloxy)[1,2,4]triazolo[4,3-b]pyridazin-3-yl]sulfanyl}-5-fluoro-1,3-benzothiazol-2-yl)-2-(4-cyclopropylpiperazin-1-yl)acetamide
N-{6-[(6-ethoxy[1,2,4]triazolo[4,3-b]pyridazin-3-yl)sulfanyl]-5-fluoro-1,3-benzothiazol-2-yl}-2-(4-ethylpiperazin-1-yl)acetamide
N-(6-{[6-(cyclobutyloxy)[1,2,4]triazolo[4,3-b]pyridazin-3-yl]sulfanyl}-5-fluoro-1,3-benzothiazol-2-yl)-2-(4-ethylpiperazin-1-yl)acetamide
2-(4-cyclopropylpiperazin-1-yl)-N-(5-fluoro-6-{[6-(oxetan-3-yloxy)[1,2,4]triazolo[4,3-b]pyridazin-3-yl]sulfanyl}-1,3-benzothiazol-2-yl)acetamide
2-(4-cyclopropylpiperazin-1-yl)-N-(5-fluoro-6-{[6-(tetrahydrofuran-3-yloxy)[1,2,4]triazolo[4,3-b]pyridazin-3-yl]sulfanyl}-1,3-benzothiazol-2-yl)acetamide
   and also the addition salts with pharmaceutically acceptable mineral and organic acids or with pharmaceutically acceptable mineral and organic bases of the said products of formula (I).

The invention also relates to pharmaceutical compositions containing, as active principle, at least one of the products of formula (I) as defined above or a pharmaceutically acceptable salt of this product or a prodrug of this product and, where appropriate, a pharmaceutically acceptable support.

The invention thus covers pharmaceutical compositions containing, as active principle, at least one of the medicaments as defined above.

Such pharmaceutical compositions of the present invention may also, where appropriate, contain active principles of other antimitotic medicaments, especially such as those based on taxol, cisplatin, DNA-intercalating agents and the like.

These pharmaceutical compositions may be administered orally, parenterally or locally as a topical application to the skin and mucous membranes or via intravenous or intramuscular injection.

These compositions may be solid or liquid and may be in any pharmaceutical form commonly used in human medicine, for instance simple or sugar-coated tablets, pills, lozenges, gel capsules, drops, granules, injectable preparations, ointments, creams or gels; they are prepared according to the usual methods. The active principle may be incorporated therein with excipients usually used in these pharmaceutical compositions, such as talc, gum arabic, lactose, starch, magnesium stearate, cocoa butter, aqueous or non-aqueous vehicles, fatty substances of animal or plant origin, paraffin derivatives, glycols, various wetting agents, dispersants or emulsifiers, and preserving agents.

The usual dosage, which is variable according to the products used, the patient treated and the complaint under consideration, may be, for example, from 0.05 to 5 g per day or preferably from 0.1 to 2 g per day for an adult.

A subject of the present invention is also the use of the products of formula (I) as defined above or of pharmaceutically acceptable salts of these products for the preparation of a medicament for inhibiting the activity of a kinase protein.

A subject of the present invention is also the use of products of formula (I) as defined above for the preparation of a medicament for treating or preventing a disease characterized by deregulation of the activity of a kinase protein.

Such a medicament may especially be intended for treating or preventing a disease in a mammal.

A subject of the present invention is also the use defined above, in which the kinase protein is a tyrosine kinase protein.

A subject of the present invention is also the use defined above, in which the tyrosine kinase protein is MET or mutant forms thereof.

A subject of the present invention is also the use defined above, in which the kinase protein is in a cell culture.

A subject of the present invention is also the use defined above, in which the kinase protein is in a mammal.

A subject of the present invention is especially the use of a product of formula (I) as defined above for the preparation of a medicament for preventing or treating diseases associated with an uncontrolled proliferation.

A subject of the present invention is particularly the use of a product of formula (I) as defined above for the preparation of a medicament for treating or preventing a disease chosen from the following group: blood vessel proliferation disorders, fibrotic disorders, “mesangial” cell proliferation disorders, metabolic disorders, allergies, asthmas, thromboses, nervous system diseases, retinopathy, psoriasis, rheumatoid arthritis, diabetes, muscle degeneration and cancers.

A subject of the present invention is thus most particularly the use of a product of formula (I) as defined above for the preparation of a medicament for treating or preventing oncology diseases and especially for treating cancers.

Among these cancers, attention is focused on the treatment of solid or liquid tumours and the treatment of cancers that are resistant to cytotoxic agents.

The cited products of the present invention may be used especially for treating primary tumours and/or metastases, in particular in stomach, liver, kidney, ovarian, bowel or prostate cancer, lung cancer (NSCLC and SCLC), glioblastomas, thyroid, bladder or breast cancers, melanomas, lymphoid or myeloid haematopoietic tumours, sarcomas, brain cancers, cancer of the larynx, cancer of the lymphatic system, bone cancers and pancreatic cancers.

A subject of the present invention is also the use of the products of formula (I) as defined above for the preparation of medicaments intended for cancer chemotherapy.

Such medicaments intended for cancer chemotherapy may be used alone or in combination.

The products of the present patent application may especially be administered alone or in combination with chemotherapy or radiotherapy or alternatively in combination, for example, with other therapeutic agents.

Such therapeutic agents may be commonly used antitumour agents.

Kinase inhibitors that may be mentioned include butyrolactone, flavopiridol and 2-(2-hydroxyethylamino)-6-benzylamino-9-methylpurine, known as olomoucine.

A subject of the present invention is also, as novel industrial products, the synthetic intermediates of formulae M1, M2, M3 and N with Rb representing a fluorine atom F, as defined above and recalled hereinbelow:

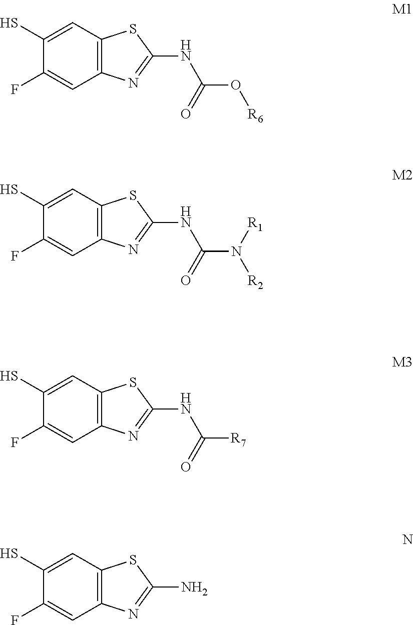

in which the groups CONR1R2, COR6 and COR7, which constitute W, may take the values of W as defined above for the products of formulae (I′) and (I″), when W≠H.

The examples that follow, which are products of formula (I), illustrate the invention without, however, limiting it.

### Experimental Section

The nomenclature of the compounds of the present invention was produced with the ACDLABS software version 11.0.

Microwave oven used:
Biotage, Initiator EXP-EU, 300 W max, 2450 MHz

The 400 MHz and 300 MHz 1H NMR spectra were acquired using a Brüker Avance DRX-400 or Brüker Avance DPX-300 spectrometer with the chemical shifts (δ in ppm) in the solvent dimethyl sulfoxide-d₆ (DMSO-d₆) referenced to 2.5 ppm, at a temperature of 303 K.

The Mass spectra were acquired either by analysis:

LC-MS-DAD-ELSD (MS=Waters ZQ)

LC-MS-DAD-ELSD (MS=Platform II Waters Micromass)

HPLC-MS-DAD-ELSD (MS=Quattro Premier XE Waters)

DAD wavelength considered λ=210-400 nm
ELSD: Sedere SEDEX 85; nebulization temperature=35° C.; nebulization pressure=3.7 bar

### EXAMPLE 1

### 1-(6-{[6-(Cyclohexyloxy)[1,2,4]triazolo[4,3-b]pyridazin-3-yl]sulfanyl}-5-fluoro-1,3-benzothiazol-2-yl)-3-[2-(morpholin-4-yl)ethyl]urea

a) 1-(6-{[6-(Cyclohexyloxy)[1,2,4]triazolo[4,3-b]pyridazin-3-yl]sulfanyl}-5-fluoro-1,3-benzothiazol-2-yl)-3-[2-(morpholin-4-yl)ethyl]urea may be prepared in the following manner:

a stream of argon is bubbled for 5 minutes through a mixture of 230 mg of N,N″-[disulfanediylbis(5-fluoro-1,3-benzothiazole-6,2-diyl)]bis{3-[2-(morpholin-4-yl)ethyl]urea} in 15 cm³ of ethanol. 3 mg of potassium dihydrogen phosphate in 0.1 cm³ of water, 310 mg of DL-dithiothreitol and 170 mg of 3-chloro-6-(cyclohexyloxy)[1,2,4]triazolo[4,3-b]pyridazine (1e) are then added. The reaction mixture is heated at 80° C. for 44 hours and then concentrated to dryness under reduced pressure. The residue is taken up in water containing sodium bicarbonate, and extracted with ethyl acetate. The organic phase is concentrated under reduced pressure. The white solid obtained is purified on silica by solid deposition, eluting with a 95/5 to 75/25 gradient of dichloromethane/(38 dichloromethane/17 methanol/2 aqueous ammonia). 253 mg of 1-(6-{[6-(cyclohexyloxy)[1,2,4]triazolo[4,3-b]pyridazin-3-yl]sulfanyl}-5-fluoro-1,3-benzothiazol-2-yl)-3-[2-(morpholin-4-yl)ethyl]urea are thus obtained in the form of a white powder, the characteristics of which are as follows:

¹H NMR SPECTRUM (400 MHz, DMSO-d₆) δ ppm 1.12-1.31 (m, 3H) 1.32-1.44 (m, 2H) 1.44-1.55 (m, 1H) 1.56-1.67 (m, 2H) 1.73-1.88 (m, 2H) 2.35-2.45 (m, 6H) 3.21-3.27 (m, 2H) 3.59 (t, J=4.4 Hz, 4H) 4.57-4.77 (m, 1H) 6.75 (t, J=4.9 Hz, 1H) 7.01 (d, J=9.8 Hz, 1H) 7.53 (d, J=10.3 Hz, 1H) 8.00 (d, J=7.3 Hz, 1H) 8.27 (d, J=9.8 Hz, 1H) 10.99 (br. s., 1H)

MASS SPECTRUM: Waters UPLC-SQD: MH+ m/z=573+; MH− =571−

b) N,N″-[Disulfanediylbis(5-fluoro-1,3-benzothiazole-6,2-diyl)]bis{3-[2-(morpholin-4-yl)ethyl]urea} may be prepared in the following manner:

0.24 cm³ of 2-(morpholin-1-yl)ethanamine and 0.39 cm³ of triethylamine are added to 322 mg of phenyl (5-fluoro-6-thiocyanato-1,3-benzothiazol-2-yl)carbamate in 10 cm³ of tetrahydrofuran at 20° C. The reaction medium is heated at 60° C. for 5 hours. The clear brown solution obtained is evaporated to dryness under reduced pressure. The residue is chromatographed on Biotage Isolera Four 12/25 (KP-SIL, 60 Å; 32-63 μM), eluting with a 99/1 to 75/25 gradient of dichloromethane/(38 dichloromethane/17 methanol/2 aqueous ammonia). 231 mg of N,N″-[disulfanediylbis(5-fluoro-1,3-benzothiazole-6,2-diyl)]bis{3-[2-(morpholin-4-yl)ethyl]urea} are thus obtained in the form of a whitish powder, the characteristics of which are as follows:

MASS SPECTRUM: Waters UPLC-SQD: MH+ m/z=711+; MH− =709−

c) Phenyl (5-fluoro-6-thiocyanato-1,3-benzothiazol-2-yl)carbamate may be prepared in the following manner:

0.75 cm³ of phenyl chlorocarbonate is added to 451 mg of 2-amino-5-fluoro-1,3-benzothiazol-6-yl thiocyanate in 5 cm³ of pyridine at 20° C. After 5 hours 30 minutes, the yellow suspension is concentrated to dryness under reduced pressure. The residue is chromatographed on Biotage Quad 12/25 (KP-SIL, 60 Å; 32-63 μM), eluting with a gradient of from 100% dichloromethane to 90/10 dichloromethane/methanol. 570 mg of phenyl (5-fluoro-6-thiocyanato-1,3-benzothiazol-2-yl)carbamate are thus obtained in the form of a beige-coloured powder, the characteristics of which are as follows:

MASS SPECTRUM: Waters ZQ: MH+ m/z=346+

d) 2-Amino-5-fluoro-1,3-benzothiazol-6-yl thiocyanate may be prepared in the manner described by K. Papke and R. Pohloudek-Fabini in Pharmazie; GE; 22, 5 1967, pp. 229-233.

e) 3-Chloro-6-(cyclohexyloxy)[1,2,4]triazolo[4,3-b]pyridazine may be prepared in the following manner:

762 mg of sodium hydride at 60% in oil are added to a solution of 3.18 g of cyclohexanol in 30 cm³ of tetrahydrofuran, at 0° C. under argon. After stirring for 15 minutes, 3 g of 3,6-dichloro[1,2,4]triazolo[4,3-b]pyridazine (commercial) are added. The brown suspension is stirred for 22 hours while allowing it to warm gradually to 20° C. The reaction mixture is poured into ice-water and the mixture is extracted with ethyl acetate. After concentrating the organic phase to dryness under vacuum, a brown oil is obtained. The oily residue is chromatographed on Biotage Quad 12/25 (KP-SIL, 60A; 32-63 μM), eluting with a 95/5 to 65/35 gradient of cyclohexane/ethyl acetate. 2.7 g of 3-Chloro-6-(cyclohexyloxy)[1,2,4]triazolo[4,3-b]pyridazine are thus obtained in the form of a yellowish powder, the characteristics of which are as follows:

### MASS SPECTRUM; LC/MS Electrospray on WATERS UPLC-SQD:

### MH+=253+

### EXAMPLE 2

### 2-Methylpropan-2-yl (5-fluoro-6-{[6-(4-fluorophenyl)[1,2,4]triazolo[4,3-b]pyridazin-3-yl]sulfanyl}-1,3-benzothiazol-2-yl)carbamate

a) 2-Methylpropan-2-yl (5-fluoro-6-{[6-(4-fluorophenyl)[1,2,4]triazolo[4,3-b]pyridazin-3-yl]sulfanyl}-1,3-benzothiazol-2-yl)carbamate may be prepared in a manner similar to that of Example 1a, but starting with 131 mg of 2-methylpropan-2-yl (5-fluoro-6-thiocyanato-1,3-benzothiazol-2-yl)carbamate, 10 cm³ of degassed ethanol, 4 mg of potassium dihydrogen phosphate in 0.2 cm³ of water, 186 mg of DL-dithiothreitol and 100 mg of 3-chloro-6-(4-fluorophenyl)-1,2,4-triazolo[4,3-b]pyridazine, after 42 hours at 80° C. 33 mg of 2-methylpropan-2-yl (5-fluoro-6-{[6-(4-fluorophenyl)[1,2,4]triazolo[4,3-b]-pyridazin-3-yl]sulfanyl}-1,3-benzothiazol-211)carbamate are thus obtained in the form of a white powder, the characteristics of which are as follows;

1H NMR SPECTRUM (400 MHz, DMSO-d₆) δ ppm 1.50 (s, 9H) 7.40 (t, J=8.8 Hz, 2H) 7.65 (d, J=10.3 Hz, 1H) 8.02 (d, J=9.8 Hz, 1H) 8.10 (dd, J=8.8, 5.4 Hz, 2H) 8.26 (d, J=7.1 Hz, 1H) 8.50 (d. J=9.8 Hz, 1H) 11.97 (br. s., 1H)

MASS SPECTRUM: Waters UPLC-SQD: MH+ m/z=513+; MH− =511−

b) 2-Methylpropan-2-yl (5-fluoro-6-thiocyanato-1,3-benzothiazol-2-yl)-carbamate may be prepared in the following manner:

41 mg of dimethylamino pyridine and 348 mg of di-tert-butyl dicarbonate are added to 300 mg of 2-amino-5-fluoro-1,3-benzothiazol-6-yl thiocyanate in 5 cm³ of dichloromethane and 0.45 cm³ of triethylamine, at 20° C. After 3 hours at 20° C., the reaction mixture is poured into water, the dichloromethane is separated out by settling and the aqueous phase is extracted with ethyl acetate. The combined organic phases are concentrated under reduced pressure. The solid yellow residue is washed with ether and dried under vacuum. 343 mg of 2-methylpropan-2-yl (5-fluoro-6-thiocyanato-1,3-benzothiazol-2-yl)carbamate are thus obtained in the form of a yellow powder, the characteristics of which are as follows:

### MASS SPECTRUM: LC/MS Electrospray on WATERS UPLC-SQD: MH+=324+

c) 3-Chloro-6-(4-fluorophenyl)-1,2,4-triazolo[4,3-b]pyridazine may be prepared in the following manner:

A mixture of 4.16 g of 4-fluorophenylboronic acid, 9.37 g of barium hydroxide octahydrate, 2.20 g of [1,1′-bis(diphenylphosphino)ferrocene]dichloropalladium(II) as a complex with dichloromethane (1:1) and 5.1 g of commercial 3,6-dichloro[1,2,4]triazolo[4,3-b]pyridazine in 40 cm³ of N,N-dimethylformamide containing 10 cm³ of water is heated in a bath at 80° C. for 1.5 hours. The beige-brown suspension obtained is cooled to 20° C. and then poured into about 200 cm³ of water. The insoluble material is filtered off by suction and washed successively with water and ether, and then dried under vacuum at 20° C. The resulting beige-coloured solid is slurried in dichloromethane, filtered off by suction and dried under vacuum at 20° C. 1.24 g of 3-chloro-6-(4-fluorophenyl)-1,2,4-triazolo[4,3-b]pyridazine are thus obtained. 30 g of silica are added to the combined mother liquors and the mixture is evaporated to dryness under vacuum. This residue is deposited on a bed of 10 g of silica in a sinter funnel, and eluted with dichloromethane. An additional 1.60 g of 3-chloro-6-(4-fluorophenyl)-1,2,4-triazolo[4,3-b]pyridazine are thus recovered, the characteristics of which are as follows:

MASS SPECTRUM: LC-MS-DAD-ELSD: MH+ m/z=249+

### EXAMPLE 3

### 5-fluoro-6-{[6-(4-fluorophenyl)[1,2,4]triazolo[4,3-b]pyridazin-3-yl]sulfanyl}-1,3-benzothiazol-2-amine

5-Fluoro-6-{[6-(4-fluorophenyl)[1,2,4]triazolo[4,3-b]pyridazin-3-yl]sulfanyl}-1,3-benzothiazol-2-amine may be prepared in the following manner:

0.9 cm³ of trifluoroacetic acid (containing 10% anisole) are gradually added over 24 hours to a mixture of 157 mg of 2-methylpropan-2-yl (5-fluoro-6-{[6-(4-fluorophenyl)[1,2,4]triazolo[4,3-b]pyridazin-3-yl]sulfanyl}-1,3-benzothiazol-2-yl)carbamate in 5 cm³ of dichloromethane at 20° C., until the starting material has disappeared. The reaction mixture is concentrated under reduced pressure. The residue is purified by chromatography on Biotage Quad 12/25 (KP-SIL, 60A; 32-63 μm), eluting with a 100/0 to 50/50 gradient of dichloromethane/(dichloromethane:38/methanol: 17/aqueous ammonia:2). 67 mg of 5-fluoro-6-{[6-(4-fluorophenyl)[1,2,4]triazolo[4,3-b]pyridazin-3-yl]-sulfanyl}-1,3-benzothiazol-2-amine are thus obtained in the form of a beige-coloured powder, the characteristics of which are as follows:

1H NMR SPECTRUM (400 MHz, DMSO-d₆) δ ppm 7.26 (d, J=10.3 Hz, 1H) 7.41 (t, J=8.8 Hz, 2H) 7.84 (s, 2H) 7.94-8.06 (m, 2H) 8.11 (dd, J=8.8, 5.4 Hz, 2H) 8.48 (d, J=−9.8 Hz, 1H)

MASS SPECTRUM; Waters UPLC-SQD; MH+ m/z=413+; MH− =411−

### EXAMPLE 4

### 1-(5-fluoro-6-{[6-(4-fluorophenyl)[1,2,4]triazolo[4,3-b]pyridazin-3-yl]sulfanyl}-1,3-benzothiazol-2-yl)-3-[2-(morpholin-4-yl)ethyl]urea

a) 1-(5-Fluoro-{[6-(4-fluorophenyl)[1,2,4]triazolo[4,3-b]pyridazin-3-yl]sulfanyl}-1,3-benzothiazol-2-yl)-3-[2-(morpholin-4-yl)ethyl]urea may be prepared in the following manner:

a stream of argon is bubbled through a solution of 217 mg of N,N″-[disulfanediylbis(5-fluoro-1,3-benzothiazole-6,2-diyl)]bis{3-[2-(morpholin-4-yl)ethyl]urea} (1b) in 10 cm³ of ethanol, for 5 minutes. 3 mg of potassium dihydrogen phosphate in 0.1 cm³ of water, 282 mg of DL-dithiothreitol and 152 mg of 3-chloro-6-(4-fluorophenyl)-1,2,4-triazolo[4,3-b]pyridazine are then added. The reaction medium is heated at 80° C. for 40 hours and then concentrated to dryness under reduced pressure. The residue is purified on silica by solid deposition, eluting with a gradient of from 100% dichloromethane to 75/25 dichloromethane/(38 dichloromethane/17 methanol/2 aqueous ammonia). 104 mg of 1-(5-fluoro-6-{[6-(4-fluorophenyl)[1,2,4]triazolo[4,3-b]pyridazin-3-yl]-sulfanyl}-1,3-benzothiazol-2-yl)-3-[2-(morpholin-4-yl)ethyl]urea are thus obtained in the form of a beige-coloured powder, the characteristics of which are as follows:

1H NMR SPECTRUM (400 MHz, DMSO-d₆) δ ppm 2.28-2.47 (m, 6H) 3.30 (masked m, 2H) 3.50-3.67 (m, 4H) 6.76 (br. s., 1H) 7.40 (t, J=8.8 Hz, 2H) 7.57 (d, J=10.0 Hz, 1H) 8.02 (d, J=9.8 Hz, 1H) 8.10 (dd, J=8.9, 5.3 Hz, 2H) 8.21 (d, J=7.3 Hz, 1H) 8.50 (d, J=9.5 Hz, 1H) 11.01 (s, 1H)

MASS SPECTRUM: Waters UPLC-SQD: MH+ m/z=569+; MH− =567−

### EXAMPLE 5

### 1-(6-{[6-(cyclopropylamino)[1,2,4]triazolo[4,3-b]pyridazin-3-yl]sulfanyl}-5-fluoro-1,3-benzothiazol-2-yl)-3-[2-(morpholin-4-yl)ethyl]urea

a) 1-(6-{[6-(Cyclopropylamino)[1,2,4]triazolo[4,3-b]pyridazin-3-yl]sulfanyl}-5-fluoro-1,3-benzothiazol-2-yl)-3-[2-(morpholin-4-yl)ethyl]urea may be prepared in a manner similar to that of Example 1a, but starting with 203 mg of N,N″-[disulfanediylbis(5-fluoro-1,3-benzothiazole-6,2-diyl)]bis{3-[2-(morpholin-4-yl)ethyl]urea} (1b) in 5 cm³ of degassed ethanol, 5 mg of potassium dihydrogen phosphate in 0.5 cm³ of water, 265 mg of DL-dithiothreitol and 120 mg of 3-chloro-N-cyclopropyl[1,2,4]triazolo[4,3-b]-pyridazin-6-amine, after 18 hours at 80° C. The reaction medium is cooled to 20° C. and the precipitate is filtered off by suction and then washed with ethanol. 198 mg of 1-(6-{[6-(cyclopropylamino)[1,2,4]triazolo[4,3-b]pyridazin-3-yl]sulfanyl}-5-fluoro-1,3-benzothiazol-2-yl)-3-[2-(morpholin-4-yl)ethyl]urea are thus obtained in the form of a cream-white powder, the characteristics of which are as follows:

¹H NMR SPECTRUM (400 MHz, DMSO-d₆) δ ppm 0.32-0.45 (m, 2H) 0.59-0.73 (m, 2H) 2.51-2.56 (masked m, 1H) 3.11 (br. s., 2H) 3.19-3.29 (masked m, 2H) 3.43-3.62 (m, 4H) 3.64-3.81 (m, 2H) 3.98 (m, J=10.5 Hz, 2H) 6.77 (d, J=9.8 Hz, 1H) 7.22 (br. s., 1H) 7.55 (d, J=10.3 Hz, 1H) 7.70 (d, J=2.7 Hz, 1H) 7.93 (d, J=9.8 Hz, 1H) 8.14 (d, J=7.3 Hz, 1H) 10.12 (br, s., 1 H) 11.39 (br. s., 1H)

MASS SPECTRUM: Waters ZQ: MH+ m/z=530+; MH− =528−

b) 3-Chloro-N-cyclopropyl[1,2,4]triazolo[4,3-b]pyridazin-6-amine may be prepared in a manner similar to that of Example 1e, but starting with 1 cm³ of cyclopropylamine and 2 g of 3,6-dichloro[1,2,4]triazolo[4,3-b]pyridazine (commercial) in 20 cm³ of N,N-dimethylformamide containing 2.5 cm³ of triethylamine, at 20° C. for 18 hours. 1.83 g of 3-chloro-N-cyclopropyl[1,2,4]triazolo[4,3-b]pyridazin-6-amine are thus obtained in the form of a white powder, the characteristics of which are as follows:

MASS SPECTRUM: Waters ZQ: Retention time Tr (min)=2.66; MH+ m/z=210+; MH− =208−

### EXAMPLE 6

### 1-(6-{[6-(cyclohexylamino)[1,2,4]triazolo[4,3-b]pyridazin-3-yl]sulfanyl}-5-fluoro-1,3-benzothiazol-2-yl)-3-[2-(morpholin-4-yl)ethyl]urea

a) 1-(6-{[6-(Cyclohexylamino)[1,2,4]triazolo[4,3-b]pyridazin-3-yl]sulfanyl}-5-fluoro-1,3-benzothiazol-2-yl)-3-[2-(morpholin-4-yl)ethyl]urea may be prepared in a manner similar to that of Example 1a, but starting with 226 mg of N,N″-[disulfanediylbis(5-fluoro-1,3-benzothiazole-6,2-diyl)]bis{3-[2-(morpholin-4-yl)ethyl]urea} (1b) in 5 cm³ of degassed ethanol, 5 mg of potassium dihydrogen phosphate in 0.5 cm³ of water, 265 mg of DL-dithiothreitol and 160 mg of 3-chloro-N-cyclohexyl[1,2,4]triazolo[4,3-b]-pyridazin-6-amine, after 18 hours at 80° C. The reaction medium is cooled to 20° C. and the precipitate is filtered off by suction and then washed with ethanol. 189 mg of 1-(6-{[6-(cyclohexylamino)[1,2,4]triazolo[4,3-b]pyridazin-3-yl]sulfanyl}-5-fluoro-1,3-benzothiazol-2-yl)-3-[2-(morpholin-4-yl)ethyl]urea are thus obtained in the form of a cream-white powder, the characteristics of which are as follows:

¹H NMR SPECTRUM (400 MHz, DMSO-d₆) δ ppm 100-1.32 (m, 5H) 1.47-1.69 (m, 3H) 1.79 (d, J=11.5 Hz, 2H) 2.36-2.46 (m, 6H) 3.22-3.28 (m, 2 H) 3.32-3.42 (m, 1H) 3.59 (t, J=4.4 Hz, 4H) 6.75 (br. s., 1H) 6.78 (d, J=10.0 Hz, 1H) 7.25 (d, J=7.1 Hz, 1H) 7.50 (d, J=10.3 Hz, 1H) 7.90 (d, J=9.8 Hz, 1H) 7.95 (d, J=7.3 Hz, 1H) 10.97 (br. s., 1H)

MASS SPECTRUM: Waters UPLC-SQD: MH+ m/z=572+; MH− =570−

b) 3-Chloro-N-cyclohexyl[1,2,4]triazolo[4,3-b]pyridazin-6-amine may be prepared in a manner similar to that of Example 1e, but starting with 3.4 cm³ of cyclohexylamine and 5 g of 3,6-dichloro[1,2,4]triazolo[4,3-b]pyridazine (commercial) in 50 cm³ of N,N-dimethylformamide containing 11.2 cm³ of triethylamine, at 20° C. for 18 hours and then at 50° C. for 4 hours. 4.45 g of 3-chloro-N-cyclohexyl[1,2,4]triazolo[4,3-b]pyridazin-6-amine are thus obtained in the form of a white powder, the characteristics of which are as follows:

MASS SPECTRUM: Waters UPLC-SQD: Retention time Tr (min)=0.86; MH+ m/z=252+; MH− =250−

### EXAMPLE 7

### 2-(4-cyclopropylpiperazin-1-yl)-N-{6-[(6-ethoxy[1,2,4]triazolo[4,3-b]pyridazin-3-yl)sulfanyl]-5-fluoro-1,3-benzothiazol-2-yl}acetamide

a) 2-(4-Cyclopropylpiperazin-1-yl)-N-{6-[(6-ethoxy[1,2,4]triazolo[4,3-b]pyridazin-3-yl)sulfanyl]-5-fluoro-1,3-benzothiazol-2-yl}acetamide may be prepared in a manner similar to that of Example 2a, but starting with 189 mg of 2-{[(4-cyclopropylpiperazin-1-yl)acetyl]amino}-5-fluoro-1,3-benzothiazol-6-yl thiocyanate (7b), 5 cm³ of degassed ethanol, 5 mg of potassium dihydrogen phosphate in 0.1 cm³ of water, 222 mg of DL-dithiothreitol and 96 mg of 3-chloro-6-ethoxy[1,2,4]triazolo[4,3-b]pyridazine (7c), after 21 hours 30 minutes at 90° C. 114 mg of 2-(4-cyclopropylpiperazin-1-yl)-N-{6-[(6-ethoxy[1,2,4]triazolo[4,3-b]pyridazin-3-yl)sulfanyl]-5-fluoro-1,3-benzothiazol-2-yl}acetamide are thus obtained in the form of a white powder, the characteristics of which are as follows:

¹H NMR spectrum (400 MHz, δ in ppm, DMSO-d₆): 0.27 (m, 2H); 0.39 (m, 2H); 1.27 (t, J=7.1 Hz, 3H); 1.61 (m, 1H); 2.42 to 2.58 (partially masked m, 8H); 3.32 (s, 2H); 4.24 (q, J=7.1 Hz, 2H); 7.06 (d, J=9.8 Hz, 1H); 7.68 (d, J=10.0 Hz, 1H); 8.22 (d, J=7.1 Hz, 1H); 8.28 (d, J=9.8 Hz, 1H)

### MASS SPECTRUM: Waters UPLC-SQD: [M+H]+: m/z 529; [M−H]−: m/z 527

b) 2-{[(4-Cyclopropylpiperazin-1-yl)acetyl]amino}-5-fluoro-1,3-benzothiazol-6-yl thiocyanate may be prepared in the following manner:

a mixture of 1.34 g of the potassium salt of (4-cyclopropylpiperazin-1-yl)acetic acid (7d) in 10 cm³ of a 2N solution of hydrogen chloride in ether is stirred for 1 hour at 20° C. The resulting suspension is evaporated to dryness under vacuum. 15 cm³ of pyridine, 226 mg of 2-amino-5-fluoro-1,3-benzothiazol-6-yl thiocyanate (1d) and 1.92 g of N-(3-dimethylaminopropyl)-N′-ethyl-carbodiimide hydrochloride are added, at 20° C., to the white residue obtained. After 2 hours 30 minutes, the brown reaction medium is evaporated to dryness. The oily residue is taken up in water. The precipitate formed is filtered off and the aqueous filtrate is then extracted with a 90/10 mixture of ethyl acetate and methanol. The filtered precipitate is taken up in ethyl acetate and then combined with the organic phase. The resulting solution is dried over magnesium sulfate, filtered and then evaporated to dryness under vacuum. The brown residue is purified on SPOT II by chromatography on a silica cartridge (SVF D26 Si60; 15-40 μM; 25 g) eluting with a 97.4/2.6 to 90/10 gradient of dichloromethane/methanol. 191 mg of 2-{[(4-cyclopropyl-piperazin-1-yl)acetyl]amino}-5-fluoro-1,3-benzothiazol-6-yl thiocyanate are thus obtained in the form of a yellow powder, the characteristics of which are as follows:

MASS SPECTRUM: Waters ZQ: Retention time Tr (min)=2.75;

### [M+H]+: m/z 392; [M−H]−: m/z 390

c) 3-Chloro-6-ethoxy[1,2,4]triazolo[4,3-b]pyridazine may be prepared in a manner similar to that of Example 1e, but starting with 19.7 g of a solution of sodium ethoxide at 21% in ethanol and 10 g of 3,6-dichloro[1,2,4]triazolo[4,3-b]pyridazine (commercial) in 100 cm³ of dioxane, after refluxing for 7 hours. 9.6 g of 3-chloro-6-ethoxy[1,2,4]triazolo[4,3-b]pyridazine are thus obtained in the form of a beige-coloured powder, the characteristics of which are as follows:

MASS SPECTRUM: Waters ZQ: Retention time Tr (min)=2.89; [M+H]⁺: m/z 199

d) The potassium salt of (4-cyclopropylpiperazin-1-yl)acetic acid may be prepared in a manner similar to the conditions described by D. T. Witiak et al.; J. Med. Chem., 1985, 28, 1228, but with 1 g of bromoacetic acid and 3 g of 4-cyclopropylpiperazine hydrochloride. 2.66 g of the potassium salt of (4-cyclopropylpiperazin-1-yl)acetic acid are thus obtained in the form of a beige-coloured powder, the characteristics of which are as follows:

MASS SPECTRUM: Waters UPLC-SQD: Retention time Tr (min)=0.10; [M+H]⁺: m/z 185

### EXAMPLE 8

### N-(6-{[6-(cyclobutyloxy)[1,2,4]triazolo[4,3-b]pyridazin-3-yl]sulfanyl}-5-fluoro-1,3-benzothiazol-2-yl)-2-(4-cyclopropylpiperazin-1-yl)acetamide

a) N-(6-{[6-(Cyclobutyloxy)[1,2,4]triazolo[4,3-b]pyridazin-3-yl]sulfanyl}-5-fluoro-1,3-benzothiazol-2-yl)-2-(4-cyclopropylpiperazin-1-yl)acetamide may be prepared in a manner similar to that of Example 2a, but starting with 266 mg of 2-{[(4-cyclopropylpiperazin-1-yl)acetyl]amino}-5-fluoro-1,3-benzothiazol-6-yl thiocyanate (7b), 5 cm³ of degassed ethanol, 5 mg of potassium dihydrogen phosphate in 0.1 cm³ of water, 315 mg of DL-dithiothreitol and 153 mg of 3-chloro-6-(cyclobutyloxy)[1,2,4]triazolo[4,3-b]pyridazine (8b), after 24 hours at 90° C. 180 mg of N-(6-{[6-(cyclobutyloxy)[1,2,4]triazolo[4,3-b]pyridazin-3-yl]sulfanyl}-5-fluoro-1,3-benzothiazol-2-yl)-2-(4-cyclopropylpiperazin-1-yl)acetamide are thus obtained in the form of a white powder, the characteristics of which are as follows:

¹H NMR spectrum (400 MHz, δ in ppm, DMSO-d₆): 0.22 to 0.30 (m, 2H); 0.36 to 0.44 (m, 2H); 1.52 to 1.65 (m, 2H); 1.69 to 1.81 (m, 1H); 1.95 to 2.11 (m, 2H); 2.19 to 2.30 (m, 2H); 2.43 to 2.58 (partially masked m, 8H); 3.32 (s, 2H); 4.86 (m, 1H); 7.05 (d, J=9.8 Hz, 1H); 7.70 (d, J=10.3 Hz, 1H); 8.15 (d, J=7.3 Hz, 1H); 8.29 (d, J=10.0 Hz, 1H); 10.15 to 14.69 (very broad m, 1H)

### MASS SPECTRUM: Waters ZQ: [M+H]⁺: m/z 555; [M−J]⁻: m/z 553

b) 3-Chloro-6-(cyclobutyloxy)[1,2,4]triazolo[4,3-b]pyridazine may be prepared in a manner similar to that of Example 1e, but starting with 10.4 cm³ of cyclobutanol, 3.17 g of sodium hydride at 60% in oil, and 10 g of 3,6-dichloro[1,2,4]triazolo[4,3-b]pyridazine (commercial) in 100 cm³ of tetrahydrofuran. 9 g of 3-chloro-6-(cyclobutyloxy)[1,2,4]triazolo[4,3-b]-pyridazine are thus obtained in the form of a beige-coloured powder, the characteristics of which are as follows:

¹H NMR spectrum (400 MHz, δ in ppm, DMSO-d₆): 1.63 to 1.96 (m, 2H); 2.07 to 2.24 (m, 2H); 2.41 to 2.52 (partially masked m, 2H); 4.95 to 5.34 (m, 1H); 7.10 (d, J=9.8 Hz, 1H); 8.28 (d, J=9.8 Hz, 1H)

### EXAMPLE 9

### N-{6-[(6-ethoxy[1,2,4]triazolo[4,3-b]pyridazin-3-yl)sulfanyl]-5-fluoro-1,3-benzothiazol-2-yl}-2-(4-ethylpiperazin-1-yl)acetamide

a) N-{6-[(6-Ethoxy[1,2,4]triazolo[4,3-b]pyridazin-3-yl)sulfanyl]-5-fluoro-1,3-benzothiazol-2-yl}-2-(4-ethylpiperazin-1-yl)acetamide may be prepared in a manner similar to that of Example 2a, but starting with 353 mg of 2-{[(4-ethyl-piperazin-1-yl)acetyl]amino}-5-fluoro-1,3-benzothiazol-6-yl thiocyanate (9b), 10 cm³ of degassed ethanol, 5 mg of potassium dihydrogen phosphate in 0.1 cm³ of water, 430 mg of DL-dithiothreitol and 185 mg of 3-chloro-6-ethoxy[1,2,4]triazolo[4,3-b]pyridazine (7c), after 21 hours at 90° C. 259 mg of N-{6-[(6-ethoxy[1,2,4]triazolo[4,3-b]pyridazin-3-yl)sulfanyl]-5-fluoro-1,3-benzothiazol-2-yl}-2-(4-ethylpiperazin-1-yl)acetamide are thus obtained in the form of a whitish powder, the characteristics of which are as follows:

¹H NMR spectrum (400 MHz, δ in ppm, DMSO-d₆): 0.98 (t, J=7.1 Hz, 3H); 1.27 (t, J=7.1 Hz, 3H); 2.31 (q, J=7.1 Hz, 2H); 2.35 to 2.44 (m, 4H); 2.48 to 2.58 (partially masked m, 4H); 3.33 (s, 2H); 4.24 (q, J=7.1 Hz, 2H); 7.06 (d, J=9.8 Hz, 1H); 7.69 (d, J=10.3 Hz, 1H); 8.23 (d, J=7.3 Hz, 1H); 8.28 (d, J=9.8 Hz, 1H); 12.16 (broad m, 1H)

MASS SPECTRUM: Waters UPLC-SQD: [M+H]+m/z 517; [M+2H]2+: m/z 259 (base peak); [M−H]−: m/z 515

b) 2-{[(4-Ethylpiperazin-1-yl)acetyl]amino}-5-fluoro-1,3-benzothiazol-6-yl thiocyanate may be prepared in a manner similar to that of Example 7b, but starting with 4.6 g of (4-ethylpiperazin-1-yl)acetic acid (commercial or the potassium salt of (4-ethylpiperazin-1-yl)acetic acid may also be prepared in a manner similar to the conditions described by D. T. Witiak et al. in Example 7d), 1 g of 2-amino-5-fluoro-1,3-benzothiazol-6-yl thiocyanate (Id) and 8.51 g of N-(3-dimethylaminopropyl)-N′-ethylcarbodiimide hydrochloride in 50 cm³ of pyridine. 714 mg of 2-{[(4-ethylpiperazin-1-yl)acetyl]amino}-5-fluoro-1,3-benzothiazol-6-yl thiocyanate are thus obtained in the form of a yellow powder, the characteristics of which are as follows:

MASS SPECTRUM: Waters UPLC-SQD: Retention time Tr (min)=0.59; [M+H]+: m/z 380; [M−H]−: m/z 378

### EXAMPLE 10

### N-(6-{[6-(cyclobutyloxy)[1,2,4]triazolo[4,3-b]pyridazin-3-yl]sulfanyl}-5-fluoro-1,3-benzothiazol-2-O-2-(4-ethylpiperazin-1-yl)acetamide

a) N-(6-{[6-(Cyclobutyloxy)[1,2,4]triazolo[4,3-b]pyridazin-3-yl]sulfanyl}-5-fluoro-1,3-benzothiazol-2-yl)-2-(4-ethylpiperazin-1-yl)acetamide may be prepared in a manner similar to that of Example 2a, but starting with 355 mg of 2-{[(4-ethylpiperazin-1-yl)acetyl]amino}-5-fluoro-1,3-benzothiazol-6-yl (5-fluoro-6-thiocyanato thiocyanate (9b), 10 cm³ of degassed ethanol, 5 mg of potassium dihydrogen phosphate in 0.1 cm³ of water, 430 mg of DL-dithiothreitol and 210 mg of 3-chloro-6-(cyclobutyloxy)[1,2,4]triazolo[4,3-b]pyridazine (8b), after 21 hours at 90° C. 232 mg of N-(6-{[6-(cyclobutyloxy)[1,2,4]triazolo[4,3-b]pyridazin-3-yl]sulfanyl}-5-fluoro-1,3-benzothiazol-2-yl)-2-(4-ethylpiperazin-1-yl)acetamide are thus obtained in the form of a white powder, the characteristics of which are as follows:

¹H NMR spectrum (400 MHz, δ in ppm, DMSO-d₆): 0.98 (t, J=7.1 Hz, 3H); 1.51 to 1.66 (m, 1H); 1.74 (m, 1H); 1.95 to 2.10 (m, 2H); 2.19 to 2.27 (m, 2 H); 2.31 (q, J=7.1 Hz, 2H); 2.39 (m, 4H); 2.54 (m, 4H); 3.33 (s, 2H); 4.86 (m, 1H); 7.05 (d, J=9.8 Hz, 1H); 7.70 (d, J=10.3 Hz, 1H); 8.15 (d, J=7.2 Hz, 1H); 8.29 (d, J=9.8 Hz, 1H); 12.20 (broad m, 1H)

MASS SPECTRUM: Waters UPLC-SQD: [M+H]+: m/z 543; [M+2H]2+: m/z 272 (base peak); [M−H]−: m/z 541

### EXAMPLE 11

### 2-(4-cyclopropylpiperazin-1-yl)-N-(5-fluoro-6-{[6-(oxetan-3-yl-oxy)[1,2,4]triazolo[4,3-b]pyridazin-3-yl]sulfanyl}-1,3-benzothiazol-2-yl)acetamide

a) 2-(4-Cyclopropylpiperazin-1-yl)-N-(5-fluoro-6-{(6-(oxetan-3-yloxy)[1,2,4]triazolo[4,3-b]pyridazin-3-yl]sulfanyl}-1,3-benzothiazol-2-yl)acetamide may be prepared in a manner similar to that of Example 2a, but starting with 346 mg of 2-{[(4-cyclopropylpiperazin-1-yl)acetyl]amino}-5-fluoro-1,3-benzothiazol-6-yl thiocyanate (7b), 10 cm³ of degassed ethanol, 5 mg of potassium dihydrogen phosphate in 0.1 cm³ of water, 407 mg of DL-dithiothreitol and 200 mg of 3-chloro-6-(oxetan-3-yloxy)[1,2,4]triazolo[4,3-b]pyridazine (11b), after 22 hours at 90° C. 238 mg of 2-(4-cyclopropylpiperazin-1-yl)-N-(5-fluoro-6-{[6-(oxetan-3-yloxy)[1,2,4]triazolo[4,3-b]-pyridazin-3-yl]sulfanyl}-1,3-benzothiazol-2-yl)acetamide are thus obtained in the form of a white powder, the characteristics of which are as follows:

1H NMR spectrum (400 MHz, δ in ppm, DMSO-d₆): 0.22 to 0.29 (m, 2H); 0.36 to 0.42 (m, 2H); 1.60 (m, 1H); 2.43 to 2.60 (partially masked m, 8H); 3.32 (s, 2H); 4.48 (m, 2H); 4.71 (m, 2H); 5.36 to 5.46 (m, 1H); 7.20 (d, J=9.8 Hz, 1H); 7.71 (d, J=10.3 Hz, 1H); 8.06 (d, J=7.3 Hz, 1H) 8.38 (d, J=9.8 Hz, 1H); 12.15 (broad m, 1H)

MASS SPECTRUM: Waters UPLC-SQD: [M+H]+: m/z 557; [M+2H]2+; m/z 279 (base peak); [M−H]−; m/z 555

b) 3-Chloro-6-(oxetan-3-yloxy)[1,2,4]triazolo[4,3-b]pyridazine may be prepared in a manner similar to that of Example 1e, but starting with 1.96 g of oxetan-3-ol, 634 mg of sodium hydride at 60% in oil and 2 g of 3,6-dichloro[1,2,4]triazolo[4,3-b]pyridazine (commercial) in 20 cm³ of tetrahydrofuran. 2.04 g of 3-chloro-6-(oxetan-3-yloxy)[1,2,4]triazolo[4,3-b]pyridazine are thus obtained in the form of a whitish powder, the characteristics of which are as follows;

MASS SPECTRUM: Waters UPLC-SQD: Retention time Tr (min)=0.41; [M+H]+: m/z 227

### EXAMPLE 12

### Rac-2-(4-cyclopropylpiperazin-1-yl)-N-(5-fluoro-{[6-(tetrahydrofuran-3-yloxy)[1,2,4]triazolo[4,3-b]pyridazin-3-yl]sulfanyl}-1,3-benzothiazol-2-yl)acetamide

a) rac-2-(4-Cyclopropylpiperazin-1-yl)-N-(5-fluoro-6-{[6-(tetrahydrofuran-3-yloxy)[1,2,4]triazolo[4,3-b]pyridazin-3-yl]sulfanyl}-1,3-benzothiazol-24)-acetamide may be prepared in a manner similar to that of Example 2a, but starting with 325 mg of 2-{[(4-cyclopropylpiperazin-1-yl)acetyl]amino}-5-fluoro-1,3-benzothiazol-6-yl thiocyanate (7b), 10 cm³ of degassed ethanol, 5 mg of potassium dihydrogen phosphate in 0.1 cm³ of water, 385 mg of DL-dithiothreitol and 200 mg of rac-3-chloro-6-(tetrahydrofuran-3-yloxy)[1,2,4]triazolo[4,3-b]pyridazine (12b), after 18 hours at 90° C., 206 mg of rac-2-(4-cyclopropylpiperazin-1-yl)-N-(5-fluoro-6-{[6-(tetrahydrofuran-3-yloxy)[1,2,4]triazolo[4,3-b]pyridazin-3-yl]sulfanyl}-1,3-benzothiazol-2-yl)acetamide are thus obtained in the form of a white powder, the characteristics of which are as follows;

¹H NMR spectrum (400 MHz, δ in ppm, DMSO-d₆): 0.19 to 0.30 (m, 2H); 0.36 to 0.43 (m, 2H); 1.60 (m, 1H); 1.91 to 2.02 (m, 1H); 2.07 to 2.22 (m, 1 H); 2.42 to 2.60 (partially masked m, 8H); 3.32 (s, 2H); 3.60 to 3.84 (m, 4H); 5.27 (m, 1H); 7.08 (d, J=9.8 Hz, 1H); 7.70 (d, J=10.0 Hz, 1H); 8.14 (d, J=7.3 Hz, 1H); 8.31 (d, J=9.8 Hz, 1H); 12.17 (broad m, 1H)

### MASS SPECTRUM; Waters UPLC-SQD: [M+H]+: m/z 571; [M−H]−: m/z 569

b) rac-3-Chloro-6-(tetrahydrofuran-3-yloxy)[1,2,4]triazolo[4,3-b]pyridazine may be prepared in a manner similar to that of Example 1e, but starting with 2.33 g of rac-tetrahydrofuran-3-ol, 634 mg of sodium hydride at 60% in oil and 2 g of 3,6-dichloro[1,2,4]triazolo[4,3-b]pyridazine (commercial) in 20 cm³ of tetrahydrofuran. 2.09 g of rac-3-chloro-6-(tetrahydrofuran-3-yl-oxy)[1,2,4]triazolo[4,3-b]pyridazine are thus obtained in the form of a beige-coloured powder, the characteristics of which are as follows:

MASS SPECTRUM: Waters UPLC-SQD: Retention time Tr (min)=0.49; [M+H]+: m/z 241

### EXAMPLE 13

### Pharmaceutical Composition

Tablets corresponding to the following formula were prepared:

| | |
|---|---|
| Product of Example 1 . . . | 0.2 g |
| Excipient for a finished tablet weighing . . . | 1 g |
| (details of the excipient: lactose, talc, starch, | |
| magnesium stearate). | |

### EXAMPLE 14

### Pharmaceutical Composition

Tablets corresponding to the following formula were prepared:

| | |
|---|---|
| Product of Example 6 . . . | 0.2 g |
| Excipient for a finished tablet weighing . . . | 1 g |
| (details of the excipient: lactose, talc, | |
| starch, magnesium stearate). | |

Examples 2 and 5 are taken as examples of pharmaceutical preparation, this preparation possibly being performed, if desired, with other products illustrated in the present patent application.

### Pharmacological Section:

### Experimental Protocols

### 1) Expression and Purification of MET, Cytoplasmic Domain Expression as Baculovirus:

The recombinant DNA His-Tev-MET (956-1390) in pFastBac (invitrogen) is transfected into insect cells and, after several viral amplification steps, the final baculovirus stock is tested for expression of the protein of interest.

After infection for 72 hours at 27° C. with the recombinant virus, the SF21 cell cultures are harvested by centrifugation and the cell pellets are stored at −80° C.

### Purification:

The cell pellets are resuspended in lysis buffer (buffer A [50 mM HEPES, pH 7.5, 250 mM NaCl, Glycerol 10%, TECP 1 mM]; Roche Diagnostics EDTA-free protease inhibitor cocktail, ref. 1873580), stirred at 4° C. until homogeneous, and then mechanically lysed using a “flounce” machine.

After centrifugation, the lysis supernatant is incubated for 2 hours at 4° C. with nickel chelate resin (His-Trap 6 Fast Flow™, GE HealthCare). After washing with 20 volumes of buffer A, the suspension is packed into a column, and the proteins are eluted with a gradient of buffer B (buffer A+290 mM imidazole).

The fractions containing the protein of interest in the light of the electrophoretic analysis (SDS PAGE) are pooled, concentrated by ultrafiltration (10 kDa cut-off) and injected onto an exclusion chromatography column (Superdex™ 200, GE HealthCare) equilibrated with buffer A.

After enzymatic cleavage of the Histidine tag, the protein is reinjected onto a new IMAC Nickel Chelate chromatography column (His-Trap 6 Fast Flow™, GE HealthCare) equilibrated with buffer A. The fractions eluted with a gradient of buffer B and containing the protein of interest after electrophoresis (SDS PAGE) are finally pooled and stored at −80° C.

For the production of autophosphorylated protein, the preceding fractions are incubated for 1 hour at room temperature after addition of ATP 2 mM, MgCl₂ 2 mM, and Na₃VO₄ 4 mM. After stopping the reaction with 5 mM of EDTA, the reaction mixture is injected onto a HiPrep desalting column (GE HealthCare) pre-equilibrated with buffer A+Na₃VO₄ 4 mM, and the fractions containing the protein of interest (SDS PAGE analysis) are pooled and stored at −80° C. The degree of phosphorylation is checked by mass spectrometry (LC-MS) and by peptide mapping.

### II) Tests A and B

A) Test A: HTRF MET test in 96-well format

In a final volume of 50 μl of enzymatic reaction, MET 5 nM final is incubated in the presence of the test molecule (for a final concentration range of 0.17 nM to 10 μM, DMSO 3% final) in MOPS 10 mM pH 7.4, DTT 1 mM, 0.01% Tween 20 buffer. The reaction is initiated with the substrate solution to obtain final concentrations of poly-(GAT) 1 μg/ml, ATP 10 μM and MgCl₂ 5 mM. After incubation for 10 minutes at room temperature, the reaction is stopped with a mix of 30 μl to obtain a final solution of Hepes 50 mM pH 7.5, potassium fluoride 500 mM, 0.1% BSA and EDTA 133 mM in the presence of 80 ng of streptavidin 61SAXLB Cis-Bio Int. and 18 ng of anti-phosphotyrosine Mab PT66-Europium Cryptate per well. After incubation for 2 hours at room temperature, the reading is taken at two wavelengths, 620 nm and 665 nm, on a reader for the TRACE/HTRF technique and the percentage of inhibition is calculated from the 665/620 ratios.

The results obtained via this test A for the products of formula (I) illustrated in the experimental section are such that the IC50 is less than 500 nM and especially less than 100 nM,

B) Test B: Inhibition of autophosphorylation of MET; ELISA technique (pppY1230,1234,1235)

a) Cell lysates: Inoculated MKN45 cells in 96-well plates (Cell coat BD polylysine) to a rate of 20 000 cells/well in 200 μl in RPMI medium+10% FCS+1% L-glutamine. Leave to adhere for 24 hours in an incubator.

The cells are treated the day after inoculation with the products at six concentrations in duplicate for 1 hour. At least three control wells are treated with the same amount of final DMSO.

Product dilution: Stock at 10 mM in pure DMSO—range from 10 mM to 30 μM with an increment of 3 in pure DMSO—Intermediate 50-fold dilutions in the culture medium, followed by removal of 10 μl added directly to the cells (200 μl): final range from 10 000 to 30 nM.

At the end of incubation, delicately remove the supernatant and rinse with 200 μl of PBS. Next, place 100 μl of lysis buffer directly in the wells on ice and incubate at 4° C. for 30 minutes. Lysis buffer: 10 mM Tris-HCl pH 7.4, 100 mM NaCl, 1 mM EDTA, 1 mM EGTA, 1% Triton X-100, 10% glycerol, 0.1% SDS, 0.5% deoxycholate, 20 mM NaF, 2 mM Na₃VO₄, 1 mM PMSF and anti-protease cocktail.

The 100 μl of lysates are transferred into a V-bottomed polypropylene plate and ELISA is performed directly or the plate is frozen at −80° C.,

### b) ELISA PhosphoMET SicSource Kit KH00281

Add 70 μl of kit dilution buffer+30 μL of cell lysates or 30 μl of lysis buffer for the blanks to each well of the kit plate. Incubate for 2 hours with gentle rocking at room temperature.

Rinse the wells four times with 400 μl of kit washing buffer. Incubate with 100 μl of anti-phospho MET antibody for 1 hour at room temperature.

Rinse the wells four times with 400 μL of kit washing buffer. Incubate with 100 μl of anti-rabbit HRP antibody for 30 minutes at room temperature (except for the wells with chromogen alone).

Rinse the wells four times with 400 μl of kit washing buffer. Introduce 100 μl of chromogen and incubate for 30 minutes in the dark at room temperature.

Stop the reaction with 100 μl of stop solution. Take the reading without delay, at 450 nM 0.1 second on a Wallac Victor plate reader.

### C) Test C: Measurement of the Cell Proliferation Via a 14C-Thymidine Pulse

The cells are inoculated in Cytostar 96-well plates in 180 μl for 4 hours at 37° C. and 5% CO₂: HCT116 cells at a rate of 2500 cells per well in DMEM medium+10% foetal calf serum+1% L-Glutamine and MKN45 cells at a rate of 7500 cells per well in RPMI medium+10% foetal calf serum+1% L-Glutamine. After these 4 hours of incubation, the products are added in 10 μl as a 20-fold concentrated solution according to the dilution method cited for ELISA. The products are tested at 10 concentrations in duplicate from 10 000 nM to 0.3 nM with an increment of 3.

After 72 hours of treatment, add 10 μl of 14C-thymidine at 10 μCi/ml to obtain 0.1 μCi per well. The incorporation of 14C-thymidine is measured on a Micro-Beta machine (Perkin-Elmer) after 24 hours of pulse and 96 hours of treatment.

All the test steps are automated on BIOMEK 2000 or TECAN stations.

The results obtained via this test B for the products of formula (I) illustrated in the experimental section are such that the 1050 is less than 10 μM and especially less than 1 μM.

The results obtained for the products illustrated in the experimental section are given in the table of pharmacological results hereinbelow, as follows:

for test A, the + sign corresponds to less than 500 nM and the ++ sign corresponds to less than 100 nM,
for test B, the + sign corresponds to less than 500 nM and the ++ sign corresponds to less than 100 nM,
for test C, the + sign corresponds to less than 10 μM and the ++ sign corresponds to less than 1 μM.

Table of pharmacological results:

| Ex. number | test A | test B | test C |
|---|---|---|---|
| 1 | ++ | ++ | ++ |
| 2 | ++ | ++ | ++ |
| 3 | ++ | ++ | ++ |
| 4 | ++ | ++ | ++ |
| 5 | ++ | ++ | ++ |
| 6 | ++ | ++ | ++ |
| 7 | ++ | ++ | ++ |
| 8 | ++ | ++ | ++ |
| 9 | ++ | ++ | ++ |
| 10 | ++ | ++ | ++ |
| 11 | + | + | |
| 12 | to be completed | to be completed | to be completed |

1. A compound of formula (I): 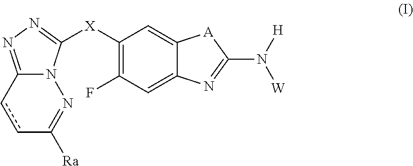
in which [symbol]
represents a single or double bond;
Ra represents a hydrogen atom; a halogen atom; an alkoxy radical optionally substituted with a chlorine atom, a hydroxyl radical or a heterocycloalkyl radical, which is itself optionally substituted; a radical —O-cycloalkyl, —O-heterocycloalkyl; —NH-cycloalkyl and —NH-heterocycloalkyl, all optionally substituted; an optionally substituted heteroaryl radical; an optionally substituted phenyl radical; a radical NHCOalkyl or NHCOcycloalkyl; or a radical NR1R2 as defined below;
X represents S, SO or SO₂;
A represents NH or S;
W represents a hydrogen atom; an alkyl, cycloalkyl or heterocycloalkyl radical optionally substituted with alkoxy, heterocycloalkyl or NR3R4; or the radical COR in which R represents:
a cycloalkyl radical or an alkyl radical, optionally substituted with a radical NR3R4, alkoxy, hydroxyl, phenyl, heteroaryl or heterocycloalkyl, which are themselves optionally substituted;
an alkoxy radical optionally substituted with NR3R4, alkoxy, hydroxyl or heterocycloalkyl; a radical O-phenyl or a radical O—(CH2)n-phenyl, with phenyl optionally substituted and n represents an integer from 1 to 4;
or the radical NR1R2 in which R1 and R2 are such that one from among R1 and R2 represents a hydrogen atom or an alkyl radical and the other from among R1 and R2 represents a hydrogen atom, a cycloalkyl radical or an alkyl radical optionally substituted with one or more radicals, which may be identical or different, chosen from the following radicals; hydroxyl, alkoxy, heteroaryl, heterocycloalkyl, NR3R4, phenyl, optionally substituted; or alternatively R1 and R2 form, with the nitrogen atom to which they are attached, a 3- to 10-membered cyclic radical optionally containing one or more other heteroatoms chosen from O, S, N and NH, this radical, including the possible NH it contains, being optionally substituted;
with R3 and R4, which may be identical or different, representing a hydrogen atom, an alkyl radical, a cycloalkyl radical, a heteroaryl radical or a phenyl radical, all optionally substituted with one or more radicals, which may be identical or different, chosen from the following radicals: hydroxyl, alkoxy, heteroaryl, heterocycloalkyl, NH2, NHalk, N(alk)2, phenyl, optionally substituted;
or alternatively R3 and R4 form, with the nitrogen atom to which they are attached, a 3- to 10-membered cyclic radical optionally containing one or more other heteroatoms chosen from O, S, N and NH, this radical, including the possible NH it contains, being optionally substituted;
all the alkyl, cycloalkyl, heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms and the following radicals: hydroxyl, oxo, alkoxy, NH2, NHalk, N(alk)2 and alkyl, cycloalkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and the following radicals: hydroxyl, oxo, alkyl and alkoxy containing from 1 to 4 carbon atoms, NH2, NHalk and N(alk)2;
racemic, enantiomeric or diastereoisomeric isomers, and pharmaceutically acceptable salts thereof.

2. The compound according to claim 1, wherein
Ra represents an alkoxy radical optionally substituted with a chlorine atom, a hydroxyl radical or a heterocycloalkyl radical, which is itself optionally substituted; a radical O-cycloalkyl; a radical NHCOalk; or a radical NR1 aR2a such that R1a and R2a represent a hydrogen atom, a cycloalkyl radical or an alkyl radical optionally substituted with one or more radicals, which may be identical or different, chosen from hydroxyl, alkoxy, heteroaryl, heterocycloalkyl, NR3R4 and phenyl radicals, optionally substituted;
and W represents a hydrogen atom; an alkyl radical optionally substituted with alkoxy, heterocycloalkyl or NR3R4; or the radical COR in which R represents:
a cycloalkyl radical or an alkyl radical optionally substituted with a radical NR3R4, alkoxy, hydroxy, phenyl, heteroaryl or heterocycloalkyl, which are themselves optionally substituted;
an alkoxy radical optionally substituted with NR3R4, alkoxy, hydroxyl or heterocycloalkyl; a radical O-phenyl or a radical O—(CH2)n-phenyl, with phenyl optionally substituted and n representing an integer from 1 to 4;
or the radical NR1R2 in which R1 and R2 are such that one from among R1 and R2 represents a hydrogen atom or an alkyl radical and the other from among R1 and R2 represents a hydrogen atom, a cycloalkyl radical or an alkyl radical optionally substituted with one or more radicals, which may be identical or different, chosen from hydroxyl, alkoxy, heteroaryl, heterocycloalkyl, NR3R4 or phenyl radicals, optionally substituted, or alternatively R1 and R2 form, with the nitrogen atom to which they are attached, a 3- to 10-membered cyclic radical optionally containing one or more other heteroatoms chosen from O, S, N and NH, this radical, including the optional NH it contains, being optionally substituted;
with R3 and R4, which may be identical or different, representing a hydrogen atom, an alkyl radical, a cycloalkyl radical, a heteroaryl radical or a phenyl radical, all optionally substituted with one or more radicals, which may be identical or different, chosen from hydroxyl, alkoxy, heteroaryl, heterocycloalkyl, NH2, NHalk, N(alk)2 or phenyl radicals, optionally substituted; or alternatively R3 and R4 form, with the nitrogen atom to which they are attached, a 3- to 10-membered cyclic radical optionally containing one or more other heteroatoms chosen from O, S, N and NH, this radical, including the optional NH it contains, being optionally substituted;
all the heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms, hydroxyl, oxo, alkoxy, NH2, NHalk, N(alk)2 radicals and alkyl, cycloalkyl, heterocycloalkyl, CH2-heterocycloalkyl, phenyl, CH2-phenyl, heteroaryl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and hydroxyl, oxo, alkyl and alkoxy radicals containing from 1 to 4 carbon atoms, NH2, NHalk and N(alk)2,
racemic, enantiomeric or diastereoisomeric isomers, and pharmaceutically acceptable salts thereof.

3. The compound according to claim 1, wherein
A represents NH or S;
W represents a hydrogen atom; an alkyl radical optionally substituted with alkoxy or heterocycloalkyl; or the radical COR in which R represents:
a cycloalkyl radical or an alkyl radical optionally substituted with a radical NR3R4, alkoxy, hydroxy, phenyl, heteroaryl, or heterocycloalkyl, which are themselves optionally substituted;
an alkoxy radical optionally substituted with NR3R4, alkoxy, hydroxyl or heterocycloalkyl; a radical O-phenyl or a radical O—(CH2)n-phenyl, with phenyl optionally substituted and n representing an integer from 1 to 4;
or the radical NR1R2, in which R1 and R2 are such that one from among R1 and R2 represents a hydrogen atom or an alkyl radical and the other from among R1 and R2 represents a hydrogen atom, a cycloalkyl radical or an alkyl radical optionally substituted with NR3R4 or with alkoxy, or alternatively R1 and R2 form, with the nitrogen atom to which they are attached, a 3- to 10-membered cyclic radical optionally containing one or more other heteroatoms chosen from O, S, N and NH, this radical, including the optional NH it contains, being optionally substituted;
with NR3R4 such that R3 and R4, which may be identical or different, represent a hydrogen atom or an alkyl radical, or alternatively R3 and R4 form, with the nitrogen atom to which they are attached, a 3- to 10-membered cyclic radical optionally containing one or more other heteroatoms chosen from O, S, N and NH, this radical, including the optional NH it contains, being optionally substituted;
the heterocycloalkyl, heteroaryl and phenyl radicals, and also the cyclic radicals that may be formed by R1 and R2 or R3 and R4 with the nitrogen atom to which they are attached, defined above, being optionally substituted with one or more radicals chosen from halogen atoms, hydroxyl, alkoxy, NH2, NHalk, N(alk)2 radicals and alkyl, heterocycloalkyl, CH2-heterocycloalkyl, phenyl, CH2-phenyl, heteroaryl, CO-phenyl and S-heteroaryl radicals, such that, in the latter radicals, the alkyl, heterocycloalkyl, phenyl and heteroaryl radicals are themselves optionally substituted with one or more radicals chosen from halogen atoms and hydroxyl, alkyl and alkoxy radicals containing from 1 to 4 carbon atoms, NH2, NHalk and N(alk)2;
racemic, enantiomeric or diastereoisomeric isomers, and pharmaceutically acceptable salts thereof.

4. The compound according to claim 1, wherein [symbol]
represents a single or double bond;
Ra represents a hydrogen atom or a halogen atom, or alternatively an optionally substituted phenyl radical;
X represents S, SO or SO₂;
A represents NH or S;
W represents a hydrogen atom or the radical COR in which R represents:
a cycloalkyl radical or an alkyl radical, optionally substituted with a radical phenyl, heteroaryl, NR3R4 or heterocycloalkyl, which are themselves optionally substituted;
an alkoxy radical optionally substituted with NR3R4, i.e. a radical O—(CH2)n—NR3R4, a radical O-phenyl or O—(CH2)n-phenyl, with phenyl optionally substituted and n represents an integer from 1 to 4;
or the radical NR1R2 in which R1 and R2 are such that one from among R1 and R2 represents a hydrogen atom or an alkyl radical and the other from among R1 and R2 represents a cycloalkyl radical or an alkyl radical optionally substituted with one or more radicals, which may be identical or different, chosen from the following radicals: hydroxyl, alkoxy, heteroaryl, heterocycloalkyl, NR3R4, phenyl, optionally substituted or alternatively R1 and R2 form, with the nitrogen atom to which they are attached, a cyclic radical optionally containing one or more other heteroatoms chosen from O, S, N and NH, this radical, including the possible NH it contains, being optionally substituted;
with R3 and R4, which may be identical or different, representing a hydrogen atom, an alkyl radical, a cycloalkyl radical, a heteroaryl radical or a phenyl radical, optionally substituted or alternatively R3 and R4 form, with the nitrogen atom to which they are attached, a cyclic radical optionally containing one or more other heteroatoms chosen from O, S, N and NH, this radical, including the possible NH it contains, being optionally substituted;
all the heterocycloalkyl, heteroaryl and phenyl radicals defined above, and also the cyclic radicals that may be formed by R1 and R2 or R3 and R4 with the nitrogen atom to which they are attached, being optionally substituted with one or more radicals chosen from halogen atoms and the following radicals: hydroxyl, oxo, alkoxy, NH2, NHalk, N(alk)2 and alkyl, cycloalkyl, CH2-heterocycloalkyl, CH2-phenyl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and the following radicals: hydroxyl, oxo, alkyl and alkoxy containing from 1 to 4 carbon atoms, NH2, NHalk and N(alk)2;
racemic, enantiomeric or diastereoisomeric isomers, and pharmaceutically acceptable salts thereof.

5. The compound according to claim 1, wherein
A represents NH or S;
W represents a hydrogen atom or an alkyl radical or the radical COR in which R represents:
an alkyl radical optionally substituted with OCH3 or NR3R4;
a cycloalkyl radical;
an alkoxy radical optionally substituted with OCH3 or NR3R4, i.e. a radical O—(CH2)n-OCH3 or a radical O—(CH2)n-NR3R4, a radical O-phenyl or O—(CH2)n-phenyl, with phenyl optionally substituted and n represents an integer from 1 to 2;
or the radical NR1R2, in which R1 and R2 are such that one from among R1 and R2 represents a hydrogen atom, a cycloalkyl radical or an alkyl radical and the other from among R1 and R2 represents an alkyl radical optionally substituted with NR3R4, or alternatively R1 and R2 form, with the nitrogen atom to which they are attached, a cyclic radical optionally containing one or more other heteroatoms chosen from O, S, N and NH, this radical, including the possible NH it contains, being optionally substituted;
with NR3R4 such that R3 and R4, which may be identical or different, represent a hydrogen atom or an alkyl radical or alternatively R3 and R4 form, with the nitrogen atom to which they are attached, a cyclic radical optionally containing one or more other heteroatoms chosen from O, S, N and NH, this radical, including the possible NH it contains, being optionally substituted;
the phenyl radicals, and also the cyclic radicals that may be formed by R1 and R2 or R3 and R4 with the nitrogen atom to which they are attached, defined above, being optionally substituted with one or more radicals chosen from halogen atoms and the following radicals: hydroxyl, alkoxy, NH2, NHalk, N(alk)2 and alkyl, CH2-heterocycloalkyl, CH2-phenyl, CO-phenyl and S-heteroaryl radicals, such that in the latter radicals, the alkyl, heterocycloalkyl, phenyl and heteroaryl radicals are themselves optionally substituted with one or more radicals chosen from halogen atoms and hydroxyl, alkyl and alkoxy radicals containing from 1 to 4 carbon atoms, NH2, NHalk and N(alk)2;
racemic, enantiomeric or diastereoisomeric isomers, and pharmaceutically acceptable salts thereof.

6. The compound according to claim 1, wherein
A represents NH or S;
W represents a hydrogen atom or the radical COR in which R represents:
an alkyl radical optionally substituted with NR3R4;
an alkoxy radical optionally substituted with NR3R4, i.e. a radical O—(CH2)n-NR3R4, a radical O-phenyl or O—(CH2)n-phenyl, with phenyl optionally substituted and n representing an integer from 1 to 2;
or the radical NR1R2, in which R1 and R2 are such that one from among R1 and R2 represents a hydrogen atom or an alkyl radical and the other from among R1 and R2 represents an alkyl radical optionally substituted with NR3R4, or alternatively R1 and R2 form, with the nitrogen atom to which they are attached, a cyclic radical optionally containing one or more other heteroatoms chosen from O, S, N and NH, this radical, including the possible NH it contains, being optionally substituted; with NR3R4 such that R3 and R4, which may be identical or different, represent a hydrogen atom or an alkyl radical or alternatively R3 and R4 form, with the nitrogen atom to which they are attached, a cyclic radical optionally containing one or more other heteroatoms chosen from O, S, N and NH, this radical, including the possible NH it contains, being optionally substituted;
the phenyl radicals, and also the cyclic radicals that may be formed by R1 and R2 or R3 and R4 with the nitrogen atom to which they are attached, defined above, being optionally substituted with one or more radicals chosen from halogen atoms and the following radicals: hydroxyl, alkoxy, NH2, NHalk, N(alk)2 and alkyl, CH2-heterocycloalkyl, CH2-phenyl, CO-phenyl and S-heteroaryl radicals, such that, in the latter radicals, the alkyl, heterocycloalkyl, phenyl and heteroaryl radicals are themselves optionally substituted with one or more radicals chosen from halogen atoms and hydroxyl, alkyl and alkoxy radicals containing from 1 to 4 carbon atoms, NH2, NHalk and N(alk)2;
racemic, enantiomeric or diastereoisomeric isomers, and pharmaceutically acceptable salts thereof.

7. The compound according to claim 1, wherein Ra represents a hydrogen atom or a chlorine atom, or the radical: 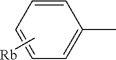
with Rb representing a halogen atom or a radical S-heteroaryl optionally substituted with a radical chosen from halogen atoms and hydroxyl, alkyl and alkoxy radicals containing from 1 to 4 carbon atoms, NH2, NHalk and N(alk)2,
racemic, enantiomeric or diastereoisomeric isomers, and pharmaceutically acceptable salts thereof.

8. The compound according to claim 1, wherein A represents NH, racemic, enantiomeric or diastereoisomeric isomers, and pharmaceutically acceptable salts thereof.

9. The compound according to claim 1, wherein A represents S, racemic, enantiomeric or diastereoisomeric isomers, and pharmaceutically acceptable salts thereof.

10. The compound according to claim 1, corresponding to formula (Ia) or (Ib): 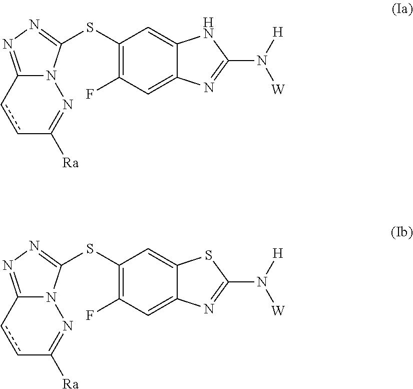
racemic, enantiomeric or diastereoisomeric isomers, and pharmaceutically acceptable salts thereof.

11. The compound according to claim 1, wherein [symbol] represents a single bond, corresponding to of formula (I′): 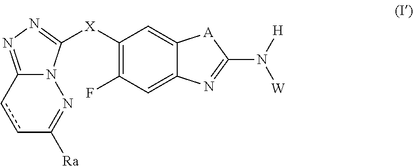
racemic, enantiomeric or diastereoisomeric isomers, and pharmaceutically acceptable salts thereof.

12. The compound according to claim 1, wherein [symbol] represents a double bond, corresponding to the products of formula (I″): 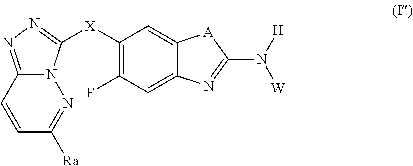
racemic, enantiomeric or diastereoisomeric isomers, and pharmaceutically acceptable salts thereof.

13. The compound of formula (Ia) according to claim 10, wherein [symbol] represents a single bond, corresponding to of formula (Ia′): 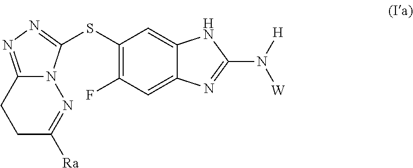
racemic, enantiomeric or diastereoisomeric isomers, and pharmaceutically acceptable salts thereof.

14. The compound of formula (Ia) according to claim 10, wherein [symbol] represents a double bond, corresponding to formula (I″a): 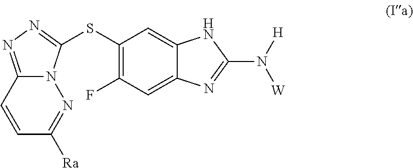
racemic, enantiomeric or diastereoisomeric isomers, and pharmaceutically acceptable salts thereof.

15. The compound of formula (Ib) according to claim 10, wherein [symbol] represents a single bond, corresponding to formula (I′b): 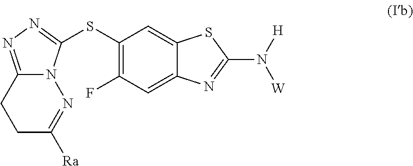
racemic, enantiomeric or diastereoisomeric isomers, and pharmaceutically acceptable salts thereof.

16. The compound of formula (Ib) according to claim 10, wherein [symbol] represents a double bond, corresponding to formula (I″b): 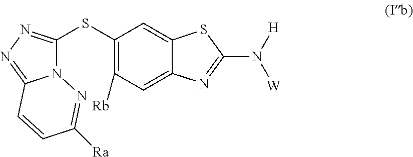
racemic, enantiomeric or diastereoisomeric isomers, and pharmaceutically acceptable salts thereof.

17. The compound according to claim 1, corresponding to the following formulae:
1-(6-{[6-(cyclohexyloxy)[1,2,4]triazolo[4,3-b]pyridazin-3-yl]sulfanyl}-5-fluoro-1,3-benzothiazol-2-yl)-3[2-(morpholin-4-yl)ethyl]urea
2-methylpropan-2-yl (5-fluoro-6-{[6-(4-fluorophenyl)[1,2,4]triazolo[4,3-b]pyridazin-3-yl]sulfanyl}-1,3-benzothiazol-2-yl)carbamate
5-fluoro-6-{[6-(4-fluorophenyl)[1,2,4]triazolo[4,3-b]pyridazin-3-yl]sulfanyl}-1,3-benzothiazol-2-amine
1-(5-fluoro-6-{[6-(4-fluorophenyl)[1,2,4]triazolo[4,3-b]pyridazin-3-yl]sulfanyl}-1,3-benzothiazol-2-yl)-3 [2-(morpholin-4-yl)ethyl]urea
1-(6-{[6-(cyclopropylamino)[1,2,4]triazolo[4,3-b]pyridazin-3-yl]sulfanyl}-5-fluoro-1,3-benzothiazol-2-yl)-3-[2-(morpholin-4-yl)ethyl]urea
1-(6-{[6-(cyclohexylamino)[1,2,4]triazolo[4,3-b]pyridazin-3-yl]sulfanyl}-5-fluoro-1,3-benzothiazol-2-yl)-3-[2-(morpholin-4-yl)ethyl]urea
2-(4-cyclopropylpiperazin-1-yl)-N-{6-[(6-ethoxy[1,2,4]triazolo[4,3-b]pyridazin-3-yl)sulfanyl]-5-fluoro-1,3-benzothiazol-2-yl}acetamide
N-(6-{[6-(cyclobutyloxy)[1,2,4]triazolo[4,3-b]pyridazin-3-yl]sulfanyl}-5-fluoro-1,3-benzothiazol-2-yl)-2-(4-cyclopropylpiperazin-1-yl)acetamide
N-{6-[(6-ethoxy[1,2,4]triazolo[4,3-b]pyridazin-3-yl)sulfanyl]-5-fluoro-1,3-benzothiazol-2-yl}-2-(4-ethylpiperazin-1-yl)acetamide
N-(6-{[6-(cyclobutyloxy)[1,2,4]triazolo[4,3-b]pyridazin-3-yl]sulfanyl}-5-fluoro-1,3-benzothiazol-2-yl)-2-(4-ethylpiperazin-1-yl)acetamide
2-(4-cyclopropylpiperazin-1-yl)-N-(5-fluoro-6-{[6-(oxetan-3-yloxy)[1,2,4]triazolo[4,3-b]pyridazin-3-yl]sulfanyl}-1,3-benzothiazol-2-yl)acetamide
2-(4-cyclopropylpiperazin-1-yl)-N-(5-fluoro-6-{[6-(tetrahydro furan-3-yloxy)[1,2,4]triazolo[4,3-b]pyridazin-3-yl]sulfanyl}-1,3-benzothiazol-2-yl)acetamide
and pharmaceutically acceptable salts thereof.

18. A process for preparing the compound according to claim 1.

19. A process for preparing the compound according to claim 1, wherein A represents NH.

20. A process for preparing the compound according to claim 1, wherein A represents S.

21. A pharmaceutical composition comprising the compound of claim 1, and pharmaceutically acceptable salts thereof.

22. A pharmaceutical composition comprising the compound according to claim 17, and pharmaceutically acceptable salts thereof.

23. A pharmaceutical composition containing, as active principle, at least one compound according to claim 1, or a pharmaceutically acceptable salt of said compound or a prodrug of said compound and a pharmaceutically acceptable support.

24. A method of inhibiting the activity of the kinase protein MET and mutant forms thereof in a patient in need thereof comprising administering to said patient a therapeutically effective amount of the pharmaceutical composition of claim 21.

25. The method according to claim 24, in which the kinase protein is in a cell culture.

26. The method according to claim 24, in which the kinase protein is in a mammal.

27. A method of treating or preventing a disease chosen from the following group: blood vessel proliferation disorders, fibrotic disorders, “mesangial” cell proliferation disorders, metabolic disorders, allergies, asthmas, thromboses, nervous system diseases, retinopathy, psoriasis, rheumatoid arthritis, diabetes, muscle degeneration and cancers in a patient in need thereof comprising administering to said patient a therapeutically effective amount of the pharmaceutical composition of claim 21.

28. A method of treating cancers in a patient in need thereof comprising administering to said patient a therapeutically effective amount of the pharmaceutical composition of claim 21.

29. The method according to claim 28, wherein solid or liquid tumours are treated.

30. The method according to claim 28, wherein said cancers are resistant to cytotoxic agents.

31. The method according to claim 28, comprising treating primary tumours and/or metastases, in particular in stomach, liver, kidney, ovarian, bowel or prostate cancer, lung cancer (NSCLC and SCLC), glioblastomas, thyroid, bladder or breast cancers, melanomas, lymphoid or myeloid haematopoietic tumours, sarcomas, brain cancers, cancer of the larynx, cancer of the lymphatic system, bone cancers and pancreatic cancers.

32. (canceled)

33. (canceled)

34. The compound according to claim 1, wherein said compound is a kinase inhibitor.

35. The compound according to claim 1, wherein said compound is a MET inhibitor.

36. A compound having one of the following formulas: 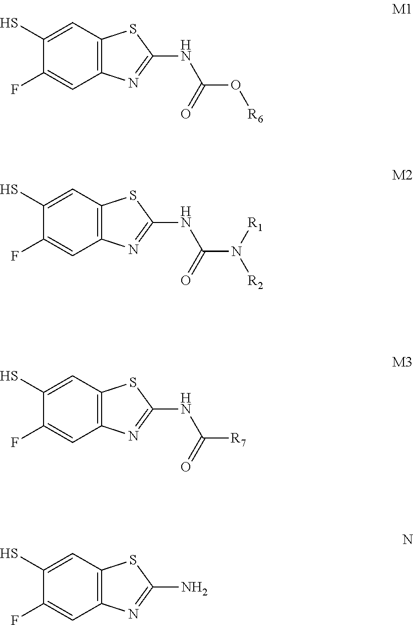
in which R6 represents an alkyl radical optionally substituted with a group NR3R4 (a radical —(CH2)n-NR3R4), alkoxy, hydroxyl, heterocycloalkyl, phenyl or —(CH2)n-phenyl, with phenyl optionally substituted and n representing an integer from 1 to 4, such that OR6 represent the corresponding values of R as defined above; R7 represents a cycloalkyl or alkyl radical optionally substituted with a radical NR3R4, alkoxy or hydroxyl or a phenyl, heteroaryl or heterocycloalkyl radical, which are themselves optionally substituted as indicated in claim 1; and Ra, R1, R2, R3 and R4 have the meanings indicated in claim 1.
